(19) United States
(12) Patent Application Publication
(10) Pub. No.: US 2013/0133101 A1
(43) Pub. Date: May 23, 2013

(54) PLANTS RESISTANT TO PATHOGENS AND METHODS FOR PRODUCTION THEREOF

(71) Applicants: Natalia Rodiuc, Mougins (FR); Yves Marco, Castenet-Tolosan (FR); Bruno Favery, Antibes (FR); Harald Keller, Vallauris (FR)

(72) Inventors: Natalia Rodiuc, Mougins (FR); Yves Marco, Castenet-Tolosan (FR); Bruno Favery, Antibes (FR); Harald Keller, Vallauris (FR)

(73) Assignee: GENOPLANTE-VALOR, PARIS (FR)

(21) Appl. No.: 13/814,380

(22) PCT Filed: Aug. 5, 2011
(86) PCT No.: PCT/EP11/63512

§ 371 Date: Feb. 5, 2013

(30) Foreign Application Priority Data
Aug. 6, 2010 (EP) 10305870.7

(51) Int. Cl. C12N 15/82

(52) U.S. Cl. CPC C12N 15/8285; C12N 15/8282; C12N 15/8281
USPC 800/276; 800/279; 800/301; 435/6.12

(57) ABSTRACT

The present invention relates to plant genes involved in negative regulation of resistance to plant pathogens and uses thereof. More particularly, the invention relates to plants having a defective phytosulfokine (PSK) function and exhibiting an increased resistance to plant pathogens. The invention also relates to methods for producing modified plants resistant to various diseases. Furthermore, the invention relates to plants having a defective PSK receptor (PSKR) function, and to methods of screening and identifying molecules that modulate PSKR expression or activity.

### FIELD OF THE INVENTION

The invention relates generally to the field of agricultural biotechnology and plant diseases. In particular, the invention relates to plant genes involved in negative regulation of resistance to plant pathogens and uses thereof. More specifically, the invention relates to plants having a defective phytosulfokine (PSK) function and exhibiting an increased resistance to plant pathogens. The invention also relates to methods for producing modified plants resistant to various diseases. Furthermore, the invention relates to plants having a defective PSK receptor (PSKR) function, and to methods of screening and identifying molecules that modulate PSKR expression or activity.

### BACKGROUND OF THE INVENTION

Plant pathogens represent a permanent threat on crop plants cultivation. In particular, infection of crop plants with bacteria, fungi, oomycetes or nematodes, can have a devastating impact on agriculture due to loss of yield and contamination of plants with toxins.

Most plant pathogenic bacteria belong to the following genera: Ralstonia, Erwinia, Pectobacterium, Pantoea, Agrobacterium, Pseudomonas, Burkholderia, Acidovorax, Xanthomonas, Clavibacter, Streptomyces, Xylella, Spiroplasma, and Phytoplasma. Plant pathogenic bacteria cause many different kinds of symptoms that include galls and overgrowths, wilts, leaf spots, specks and blights, soft rots, as well as scabs and cankers. Some plant pathogenic bacteria produce toxins or inject special proteins that lead to host cell death or produce enzymes that break down key structural components of plant cells. An example is the production of enzymes by soft-rotting bacteria that degrade the pectin layer that holds plant cells together. Still others, such as Ralstonia spp., colonize the water-conducting xylem vessels causing the plants to wilt and die. Agrobacterium species even have the ability to genetically modify or transform their hosts and bring about the formation of cancer-like overgrowths called crown gall. Bacterial diseases in plants are difficult to control. Emphasis is on preventing the spread of the bacteria rather than on curing the plant. Cultural practices can either eliminate or reduce sources of bacterial contamination, such as crop rotation to reduce over-wintering. However, the most important control procedure is ensured by genetic host resistance providing resistant varieties, cultivars, or hybrids.

Nematodes are microscopic, worm-like organisms. They most commonly feed on plant roots, but some nematodes invade leaf tissue. Nematodes suck out liquid nutrients and inject damaging materials into plants. They injure plant cells or change normal plant growth processes. Symptoms of nematodes include swelling of stems or roots, irregular branching, deformed leaves, lack of blossoming and galls on roots. Nematodes can facilitate the entry of viruses and fungi into plants. Root-knot nematodes (Meloidogyne spp.) and cyst nematodes (Globodera spp. and Heterodera spp.) are the most economically damaging genera of plant-parasitic nematodes on horticultural and field crops. Currently, nematicides are the most important means of controlling nematodes. However, most of nematicides are non-specific, notoriously toxic and pose a threat to the soil ecosystem, ground water and human health. In the context of banning of most of these compounds, novel control measures are needed.

Oomycetes are fungus-like plant pathogens that are devastating for agriculture and natural ecosystems. Phytophthora species cause diseases such as dieback, late blight in potatoes, sudden oak death, and are responsible for severe crop losses (such as 30% of the worldwide potato production). Pythium species are necrotrophs that kill plants and are responsible for pythiosis of crops, such as corn. Downy mildews, like Plasmopara viticola infecting grape, are biotrophic pathogens, which keep their hosts alive but weeken them in a way that severely affects yields. Downy mildews are easily identifiable by the appearance of white, brownish or olive “mildew” on the lower leaf surfaces. Oomycetes from the genus Albugo provoke white rust or white blister diseases on a variety of flowering plants. Oomycetes were long time considered as fungi, because they are heterotrophic, mycelium-forming organisms. However, several morphological and biochemical characteristics discriminate oomycetes from fungi. Current taxonomy clusters oomycetes with photosynthetic organisms like brown algae or diatoms within the kingdom of stramenopiles. Due to their particular physiological characteristics, no efficient treatments against diseases caused by these microorganisms are presently available. Pesticides currently used against oomycetes rely on the phenylamide metalaxyl, which inhibits RNA polymerase-1. Metalaxyl impacts the environment, and resistance of the pathogens to this oomycide develop rapidly, now being a general characteristic of pathogenic P. infestans and P. capsici populations from potato and pepper, respectively.

Common fungal diseases include powdery mildew, rust, leaf spot, blight, root and crown rots, damping-off, smut, anthracnose, and vascular wilts. Currently, fungal diseases are controlled for example by applying expensive and toxic fungicidal, chemical treatments using, e.g., probenazole, tricyclazole, pyroquilon and phthalide, or by burning infected crops. These methods are only partially successful since the fungal pathogens are able to develop resistance to chemical treatments.

To reduce the amount of pesticides used, plant breeders and geneticists have been trying to identify disease resistance loci and exploit the plant's natural defense mechanism against pathogen attack.

Plants can recognize certain pathogens and activate defense in the form of the resistance response that may result in limitation or stopping of pathogen growth. Many resistance (R) genes, which confer resistance to various plant species against a wide range of pathogens, have been identified. However, the key factors that switch these genes on and off during plant defense mechanisms remain poorly understood. Furthermore, pathogens may mutate and overcome the protection conferred by resistance genes. To control late blight disease, introgression of dominant resistance genes into susceptible cultivars has frequently been used to manage Phytophthora resistance. Eleven R genes from the wild potato species, Solanum demis sum have been introduced into modern potato cultivars. However, P. infestans races quickly evaded the new single gene-mediated resistance properties of the cultivars. R gene introgression thus has shown its limits for Phytophthora resistance breeding, and alternative programs have to be developed to render oomycete resistance durable.

Phytosulfokine (PSK) is a secreted peptide that has been first identified in the medium derived from asparagus (Asparagus officinalis L.) mesophyll culture and was proposed to be the main chemical factor responsible for “conditioning” or “nursing” i.e., the growth-promoting effects triggered by culture media previously used for cell culture or by physically separated “feeder” cells (Matsubayashi and Sakagami, 1996).

PSK peptides were also isolated from conditioned medium derived from rice (Oryza sativa L.) suspension cultures and identified to be present in two forms: a sulfated pentapeptide ([H-Tyr(SO3H)-Ile-Tyr(SO3H)-Thr-Gln-OH], PSKα) and its C-terminal-truncated tetrapeptide ([H-Tyr(SO3H)-Ile-Tyr(SO3H)-Thr-OH], PSKβ) (Matsubayashi Y. et al., 1997). The authors have suggested that a signal transduction pathway mediated by PSK peptide factors is involved in plant cell proliferation. PSK is produced from about 80 amino acids long precursor peptides via post-translational sulfation of tyrosine residues and proteolytic processing (Yang et al., 1999). Genes encoding PSK precursors are redundantly distributed in the genome and are expressed in cultured cells and in a variety of tissues, including leaves, stems, flowers and roots (Matsubayashi Y. et al., 2006; Kutschmar et al., 2008).

Two PSKR receptors have been identified in different plant species: PSKR1 and PSKR2. These receptors are members of the leucine-rich repeat receptor kinase (LRR-RK) family. PSK interacts with its receptor in a highly specific manner with a nanomolar dissociation constant. Furthermore, the PSK binding domain of carrot PSKR1 (DcPSKR1) has been identified by photoaffinity labeling (Shinohara et al., 2007). The authors have found that deletion of Glu503-Lys517 completely abolishes the ligand binding activity of DcPSKR1. This region is in the island domain flanked by extracellular LRRs, indicating that this domain forms a ligand binding pocket that directly interacts with PSK.

PSK is mainly known as an endogenously secreted, sulfated 5-amino-acid peptide that is a key factor regulating cellular dedifferentiation and redifferentiation and that affects cellular potential for growth via binding to PSK receptor (PSKR). Recently, besides the mitogenic activity, an antifungal activity of PSK peptide has been suggested by Bahyrycz et al. (2008). This document shows that the PSKα and -β peptides inhibit in vitro the mycelium growth of Phoma nareissi and Botrytis tulipae pathogens in a dose-dependent manner.

Loivamaeki et al., 2010 also propose a role of PSK signaling in wound formation in plants. Transcriptional activation of PSK/PSKR1 in crown galls is likely due to the cellular redifferentiation processes occurring during tumorigenesis. Activation of PSK signaling as a wound response has also been suggested by Motose et al., Plant Physiol. 150, 437-447, 2009.

Amano et al., 2007 concerns the identification of a new sulphated glycopeptide PSY1, related to phytosulphokines, and its involvement in developmental processes.

WO 02/083901 concerns a method of modifying growth, architecture, or morphology of a plant, based on the modulation of expression or activity of a GREP (Growth Regulating Protein) polypeptide or of a PSK homolog identified in rice, OsPSK.

PSK is thus essentially presented in the art as a regulator of cell proliferation or differentiation, with possible antifungal activity. There is no disclosure or suggestion in the art that PSK is a key regulator of pathogen resistance in plants.

### SUMMARY OF THE INVENTION

The present invention provides novel and efficient methods for producing plants resistant to pathogens. Surprisingly, the inventors have discovered that mutant plants with defective PSK and/or PSK receptor (PSKR) gene(s) are resistant to plant diseases while plants over-expressing the PSK or PSKR gene are more susceptible to plant diseases. The inventors have also demonstrated that such plants with a defective PSK or PSKR gene function acquire improved resistance to different types of pathogens, such as oomycete, nematode and bacterial pathogens, showing the broad application of this discovery.

An object of this invention therefore relates to plants comprising a defective PSK function. As will be discussed, said plants exhibit an increased or improved resistance to plant pathogens. Preferably, said plants are dicots, preferably selected from the families Solanaceae (e.g. tomato), Liliaceae (e.g. asparagus), Apiaceae (e.g. carrot), Chenopodiaceae (e.g. beet), Vitaceae (e.g. grape), Fabaceae (e.g. soybean), Cucurbitaceae (e.g. Cucumber) or Brassicacea (e.g. rapeseed, Arabidopsis thaliana), or monocots, preferably selected from the cereal family Poaceae (e.g. wheat, rice, barley, oat, rye, sorghum or maize).

The invention more particularly relates to plants having a defective PSK peptide(s) and/or PSK receptor, preferably PSKR1 receptor, and exhibiting an increased resistance to plant pathogens.

Another particular object of this invention relates to plants comprising defective PSK genes and exhibiting an increased resistance to plant pathogens.

A further particular object of this invention relates to plants comprising a defective PSKR gene and exhibiting an increased resistance to plant pathogens.

A further object of this invention relates to seeds of plants of the invention, or to plants, or descendents of plants grown or otherwise derived from said seeds.

A further object of the invention relates to a method for producing plants having increased resistance to plant pathogens, wherein the method comprises the following steps:
(a) inactivation of PSK and/or PSKR gene(s) in plant cells;
(b) optionally, selection of plant cells of step (a) with defective PSK and/or PSKR gene(s);
(c) regeneration of plants from cells of step (a) or (b); and
(d) optionally, selection of a plant of (c) with increased resistance to pathogens, said plant having defective PSK or PSKR gene(s).

As will be further disclosed in the present application, the PSK function may be rendered defective by various techniques such as for example deletion, insertion and/or substitution of one or more nucleotides, site-specific mutagenesis, ethyl methanesulfonate (EMS) mutagenesis, targeting induced local lesions in genomes (TILLING), EcoTILLING, knock-out techniques, or by gene silencing induced by RNA interference. The PSK function may also be rendered defective by altering the activity of the PSK peptide or receptor, e.g., using specific antibodies or a soluble receptor.

The invention also relates to a method for conferring or increasing resistance to plant pathogens to a plant, comprising a step of inhibiting permanently or transiently the PSK function in said plant or an ancestor thereof, e.g., by inhibiting the expression of the PSK gene(s) and/or the PSKR gene(s) in said plant.

The invention also relates to a method for protecting plants against pathogens, comprising a step of inhibiting permanently or transiently the PSK function in said plant or an ancestor thereof, e.g., by inhibiting the expression of the PSK gene(s) and/or the PSKR gene(s) in said plant.

The invention also relates to a method for decreasing pathogen proliferation in a plant, comprising a step of inhibiting permanently or transiently the PSK function in said plant or an ancestor thereof, e.g., by inhibiting the expression of the PSK gene(s) and/or the PSKR gene(s) in said plant.

Another object of this invention relates to an inhibitory nucleic acid, such as an RNAi, an antisense nucleic acid, or a ribozyme, that inhibits the expression (e.g., transcription or translation) of the PSK and/or PSKR gene(s). Another object of the invention relates to the use of such nucleic acid for increasing resistance of plants or plant cells to plant pathogens and/or for decreasing plant pathogen proliferation in plants or plant cells and/or for protecting plants or plant cells against plant pathogens.

The invention also relates to methods of identifying molecules that modulate the PSKR gene expression, the method comprising:
(a) providing a cell comprising a nucleic acid construct that comprises a PSKR gene promoter sequence operably linked to a reporter gene;
(b) contacting the cell with a candidate molecule;
(c) measuring the activity of PSKR promoter by monitoring of the expression of a marker protein encoded by the reporter gene in the cell;
(d) selecting a molecule that modulates the expression of the marker protein.

Preferably, the selected molecules inhibit the expression or the activity of PSKR, preferably PSKR1.

The invention also relates to uses of the molecules selected according to the above methods for increasing resistance of plants to plant pathogens and/or for decreasing plant pathogen proliferation in plants or plant cells and/or for protecting plants or plant cells against plant pathogens.

The invention also relates to an antibody that specifically binds a PSK peptide or receptor, or a fragment or derivative of such antibody having essentially the same antigenic specificity, as well as to the use thereof to improve or cause pathogen resistance in plants and/or for decreasing plant pathogen proliferation in plants or plant cells and/or for protecting plants or plant cells against plant pathogens.

The invention is applicable to produce legumes, vegetables and cereals having increased resistance to pathogens, and is particularly suited to produce resistant tomato, potato, asparagus, carrot, beet, rapeseed, grape, wheat, rice, barley, oat, rye, sorghum or maize.

### BRIEF DESCRIPTION OF THE DRAWINGS

FIG. 1: Constitutive expression of the PSK2 gene. The expression of the PSK2 gene (transgenic Arabidopsis line PSK2pro:GFP:GUS) is developmentally regulated. (A-E): PSK2 expression in the root system. (A,B) GUS activity (A) and GFP (B) revealing PSK2 promoter activation is detectable in the root tips (lateral root cap) but not in the elongation zone. (C,D) In fully differentiated roots, PSK2 expression localizes to the vascular cylinder. (E) Expression of PSK2 in lateral root primordial; (F-I): PSK2 expression in the shoots is localized in the vascular system of leaves and cotyledons (F), trichomes (G), and stomata (H,I). All analyses were performed on 2 week-old seedlings.

FIG. 2: PSK gene expression patterns in Arabidopsis thaliana after nematode and oomycete infection. (2A) Expression profiling of PSK genes was analyzed by microarray hybridizations. Samples were prepared from isolated galls and infected cotyledons at different time points after infection with M. incognita and H. arabidopsidis, respectively. Represented are mean Log2 ratios between infected- and uninfected tissues for two biological replicates. nc, not changed. (2B) Relative PSK transcript accumulations in Arabidopsis galls at 7 (white bars), 14 (grey bars), and 21 (black bars) days after nematode inoculation (DAI) by quantitative RT-PCR in comparison to uninfected roots. Shown is a representative experiment giving mean values (±SD) from 3 technical replicates. (2C) PSK2 expression pattern in galls of M. incognita-infected roots of the transgenic Arabidopsis line PSK2pro:GFP:GUS. A,B. Reduced GUS activity is revealed in the center of developing galls. C. GFP signal was not detected in nematode feeding cells in projections of serial confocal optical in vivo sections. *, giant cell; n, nematode.

FIG. 3: Developmentally regulated expression of the PSKR1 gene. (A) In the transgenic Arabidopsis line PSKR1pro:GFP:GUS, GUS activity revealing PSKR1 promoter activation is detectable in differentiated root tissues and the root cap, but not in the dividing and elongation zone. (B) Constitutive PSKR1 transcription in root cells, as monitored through GFP fluorescence. (C) Transcription of PSKR1 occurs in the root and the transition zone, but not in the hypocotyl. (D-E) GFP fluorescence in the epidermis of cotyledons localizes to stomata. All analyses were performed with 2 week-old seedlings.

FIG. 4: PSKR1 gene expression pattern in Arabidopsis thaliana after oomycete infection. (4A) PSKR1 transcript abundance was analyzed by qRT-PCR at different time points after spray-treatment of Arabidopsis (ecotype Ws-0) cotyledons with water, or with conidiospore suspensions at 40,000 spores/ml of the downy mildew pathogen, Hyaloperonospora arabidopsidis (Hpa). Shown are means (±SD) from 3 technical replicates normalized for values from 2 reference genes (At5g62050 and At5g10790), as calculated by the qBase1.3.5 software. The experiments performed with samples from two biological replicates gave similar tendencies. Dpi: Days post inoculation. (4B) Transcriptional activation of PSKR1 in response to Hpa infection, as monitored through the GUS reporter gene activity in the transgenic Arabidopsis line PSKR1pro:GFP:GUS. Before inoculation, constitutive expression of PSKR1 is visible through GUS activity in cotyledons at time point 0. Upon inoculation, expression increases continuously and localizes to infected areas of the mesophyll.

FIG. 5: PSKR1 gene expression pattern in Arabidopsis thaliana after nematode infection. (5A) PSKR1 transcript analysis by qRT-PCR at 7 (white bars), 14 (gray bars), and 21 (black bars) days after inoculation (DAI). Two biological replicates were performed. The bars represent mean values (±SD) from two independent experiments. (5B) Expression pattern of the GFP reporter gene under control of the PSKR1 promoter in galls of the transgenic Arabidopsis line PSKR1pro:GFP:GUS, which were induced by M. incognita in roots, 7 (A) and 21 (B) DAI with 150 surface-sterilized freshly hatched M. incognita J2 larvae.

FIG. 6: A psk3 knock-out mutant is less susceptible to oomycete infection. (6A) Schematic illustration of the genomic organization of PSK3 (locus At3g44735), primer attachment sites, and T-DNA insertion and orientation in genomic DNA from line psk3-1 (SAIL_{—}378_F03). Bars represent exons and lines correspond to introns (between exons) and untranslated sequences (at the 5′ end and at the 3′end). The T-DNA insertion localizes within the third exon. Amplicons revealing the PSK3 transcript are not detected in the mutant line, thus confirming the molecular knock-out phenotype. Amplification of the constitutively expressed EF1α gene (At1g07930) transcript showed that similar amounts of intact cDNAs were used for RT-PCR experiments. (6B) Quantitative analysis for the interaction phenotype of the PSK3 knockout mutant with H. arabidopsidis. Sporulation of H. arabidopsidis isolate Noco2 on cotyledons of the Arabidopsis psk3-1 mutant is reduced by >50%, when compared to wild-type plants (Col-0). Plantlets were collected 7 days post inoculation in 1 ml of water, vortexed, and the titer of liberated conidiospores was determined with a hemocytometer. For statistics, 20 samples at 10 plantlets were prepared for each line and analysis. The bars represent mean values (±SD). The experiment was repeated 3 times with similar results. Statistically significant differences for values compared with the wild type were determined by Student's t-test (*** P<0.0001).

FIG. 7: Over-expression of the PSK2 or PSK4 gene increases susceptibility to H. arabidopsidis, M. incognita, and R. solanacearum. (7A) Quantitative analysis for the interaction phenotype with H. arabidopsidis of transgenic lines overproducing PSK2 (Arabidopsis line p35S:PSK2) and PSK4 (Arabidopsis line p35S:PSK2). The bars represent mean values (±SD). The experiment was repeated 3 times with similar results. Statistically significant differences for values compared with the wild type were determined by Student's t-test (*** P<0.0001). (7B) Root knot nematode infection is significantly stimulated in the transgenic lines constitutively overexpressing PSKs. Arabidopsis plants were infected in vitro 14 d after germination with 150 surface-sterilized freshly hatched M. incognita J2. Statistically significant differences were determined by the Student's t test (* P<0.01, ** P<0.001, *** P<0.0001). (7C) Bacterial multiplication is strongly enhanced in transgenic lines constitutively overexpressing PSKs. Four week-old plants were root-inoculated with a solution containing 10⁷ bacteria per ml of the virulent bacterial isolate RD15. For analyzing bacterial internal growth, the aerial parts of three inoculated plants were weighed and ground in a mortar after addition of sterile water (2.0 ml per g of fresh weight). Various dilutions of the ground material were then performed with sterile water and 3×40 μl of bacterial suspensions were spotted on petri plates containing solid SMSA medium (Elphinstone et al., 1996), and grown at 30° C. For each time point, triplicate assays were performed for each A. thaliana line. The bars represent mean values (±SD).

FIG. 8: The pskr1 knock-out mutants are less susceptible to infection by H. arabidopsidis. (8A) Schematic illustration of the genomic organization of AtPSKR1 (locus At2g02220), primer attachment sites, and T-DNA insertions and orientations in genomic DNA. (8B) RT-PCR revealed PSKR1 transcripts in wild-type Arabidopsis (Col-N8846, Ws, Col-0, and Col-8 CS60000). Amplification of transcripts from the constitutively expressed AtEF1α gene (At1g07930) show that similar amounts of intact cDNAs were used for RT-PCR experiments. (8C) Allelic pskr1 mutants show reduced H. arabidopsidis sporulation. For statistics, 20 samples at 10 plantlets were prepared for each line and analysis. The bars represent mean values (±SD), and *** indicates significant differences between wild-type and mutant lines with P<0.0001, as determined by Student's t-test. All experiments were repeated 3 times and gave similar results. 1-1, 1-2, 1-3, and 1-4 represent the mutants pskr1-1, pskr1-2, pskr1-3, and pskr1-4, respectively.

FIG. 9: The pskr1 knock-out mutants are less susceptible to infection by M. incognita. The nematode infects roots and initiates gall formation to a similar extent in pskr1 mutants and wild-type plants, as analyzed 10 days post inoculation (Dpi). A reduction in the amount of mature galls is observed in pskr1 mutants at 21 Dpi. The inhibition of nematode development in the absence of PSKR1 becomes most evident during the parthenogenetic production of egg masses, which are strongly reduced on pskr1 mutants at 75 Dpi. Data represent means (±SD) from at least two experiments in which a minimum of 50 seedlings of each line were evaluated for nematode infection. *** represents statistically significant differences with P<0.0001, as determined by Student's t-test.

FIG. 10: The pskr1 knock-out mutants are less susceptible to infection by R. solanacearum. Plants with a Ws (A) and Col (B) genetic background were root-inoculated with the virulent bacterial isolates RD15 and GMI1000, respectively. Approximately 2 cm were cut from the bottom of the Jiffy pots and the exposed roots of the plants were immersed for 3 min in a suspension containing 10⁷ bacteria per ml. The plants were then transferred to a growth chamber with a day/night cycle of 8 h at 27° C., 120-140 μE m-1s-2 and 16 h at 26° C., respectively, keeping relative humidity at 75%. Disease symptoms on inoculated plants were scored at 3, 4, 5, 6, and 7 days post inoculation according to a disease index (DI) covering DI 0 (no wilt), and DI 1, DI 2, DI 3, and DI 4, representing 25%, 50%, 75%, and 100% of wilted leaves, respectively. Shown is a representative experiment among several repetitions with similar results, giving means (±SD) from inoculations of at least 28 plants/line. All pskr1 mutants are significantly less susceptible during the exponential bacterial growth phase between 3 and 5 days post inoculation with P<0.0001. The Col genetic background (B) of A. thaliana shows an overall higher susceptibility to R. solanacearum, and the effect of the pskr1 mutation is most pronounced in pskr1-2 in the Ws genetic background (A). Full susceptibility to R. solanacearum was restored through the introduction of a fully functional PSKR1 gene into the pskr1-2 genetic background (complemented Arabidopsis line Cppskr1-2, compare legend to FIG. 11). An acceleration of disease at late time points of infection was observed in the line overexpressing PSKR1 under the control of the constitutive 35S promoter (overexpressing line PSKR1-OE, compare legend to FIG. 11).

FIG. 11: Reduced susceptibility of pskr mutants is reverted by expression of a functional PSKR gene. Overexpression of the PSKR gene increases susceptibility to H. arabidopsidis. Downy mildew susceptibility correlates with PSKR1 expression (A) Conidiospores/mg FW levels obtained in the pskr1-2 mutant and transgenic lines obtained after infection with H. arabidopsidis. The mutant phenotype of pskr1-2 (Ws-0 background) is fully reverted in line Cppskr1-2 through complementation with a genomic 5,472 by fragment comprising the 1,771 bb region 5′ of the translation initiation codon, 3027 by of entire coding sequence and 650 by of 3′non-translated region of At2g02220. The genomic fragment was amplified by PCR, cloned into the Gateway destination vector pHGW (Karimi et al., 2002), and transferred into pskr1-2 by Agrobacterium-mediated transformation. Overexpression of PSKR1 in the Ws-0 wild-type (line PSKR1-OE) increases downy mildew susceptibility by almost 100%. For overexpression of the gene, 3,060 by of the coding region including Start and Stop codons were amplified from genomic DNA, cloned into the Gateway destination vector pH2GW7 (Karimi et al., 2002), and mobilized into Arabidopsis by Agrobacterium-mediated transformation. The pathogen assays were performed as described before. The bars represent mean values (±SD), and *** indicates significant differences between wild-type and mutant lines with P<0.0001, as determined by Student's t-test. All experiments were repeated 3 times and gave similar results. (B) Expression levels of PSKR1 in the different mutant and transgenic lines obtained after infection with H. arabidopsidis. Relative PSKR1 transcript accumulations in Arabidopsis seedlings (15 days after sowing) were determined by quantitative real time RT-PCR. Expression ratios were calculated using the 2⁻⁽^{ΔΔCT}) method with UBP22 (At5g10790) for normalization and wild-type PSKR1 expression as the reference. The bars (±SD) represent mean values of three technical replicates.

FIG. 12: Reduced disease susceptibility of pskr1 mutants is not a consequence of constitutively activated, or pathogen-triggered defense responses. The activation of salicylic acid (SA)-, jasmonic acid (JA)-, and ethylene (JA/ethylene)-mediated defense signaling pathways in Arabidopsis is independent of PSKR1. Marker genes for SA-, JA, and JA/ethylene-mediated signaling pathways were PR1a (At2g14610) PDF1.2 (At5g44420), and PR4 (At3g04720), respectively. Expression of these defense-related genes was analyzed by quantitative real-time RT-PCR in wild type (Ws), mutant (pskr1-2), and transgenic PSKR1 overexpressor (PSKR1-OE) plants upon spray treatment of cotyledons with water, or with conidiospore suspensions (40,000 spores/ml) of the H. arabidopsidis isolate Emwa1. Samples for RNA extraction and qRT-PCR were prepared at time point 0, and 24, 48, 72, and 120 hours after onset of treatment. Relative quantities of marker gene transcripts were normalized with AtOXA1 (At5g62050) and AtUBP22 (At5g10790) using the Q-Base software. Represented are means (±SD) from 3 technical replicates. Two independent experiments gave similar results.

FIG. 13: PSKR1 suppression causes reduced proliferation of R. solanacearum, H. arabidopsidis, and M. incognita. (A, B) Bacterial multiplication is strongly reduced in the absence of PSKR1 in the pskr1-2 mutant. For each time point, triplicate assays were performed for each A. thaliana line. The bars represent mean values (±SD). A and B are representations of the same experimental results with bacterial titers given as absolute and log values, respectively. Bacterial multiplication was drastically reduced (˜1,000-fold) in the pskr1-2 mutant, restored in the complemented line Cppskr1-2, and increased (˜2-fold) in the overexpressing line PSKR1-OE. (C) Oomycete hyphal development in leaf tissues is reduced in the absence of PSKR1 in the pskr1-2 mutant. Plants were spray-inoculated with 40,000 spores/ml and cotyledons were collected 5 days post inoculation. The development of hyphae within infected cotyledons was visualised by trypan blue staining. A fully developed, branched hyphal network was observed in the Ws wild-type plants. The network and hyphal branching was strongly reduced in the absence of PSKR1 (line pskr1-2), but became aberrant upon overexpression of PSKR1 (line PSKR1-OE). Shown are representative transmission light micrographs. (D) The reduced egg mass production by M. incognita is a consequence of reduced giant cell sizes in the absence of PSKR1. For morphological analyses, nematode-infected roots of pskr1-2, PSKR1-OE and wild-type plants (ecotype Ws) were fixed in 2% glutaraldehyde in 50 mM Pipes buffer (pH 6.9) on 7, 14 and 21 days post inoculation and then dehydrated and embedded in Technovit 7100 (Heraeus Kulzer, Wehrheim, Germany) as described by the manufacturer. Embedded tissues were sectioned (3 μm) and stained in 0.05% toluidine blue, mounted in Depex (Sigma) and microscopy was performed using bright field optics. Images were collected with a digital camera (Axiocam; Zeiss). Tissue sections through galls on 7 days post inoculation from pskr1-2 and PSKR1-OE showed no difference in gall and giant cells formation in comparison with control. At later stages of gall development (14 and 21 days post inoculation) the giant cells from pskr1-2 mutant plants were significantly smaller. For giant cell surface measurements, serial sections stained with toluidine blue were examined using the AxioVision V 4.8.1.0 software. The three biggest giant cells per gall from at least 50 galls per phenotype were chosen for measurements. Galls from pskr1-2 mutant plants contain significantly smaller giant cells in comparison to control plants at 14 days post inoculation.

FIG. 14: Representation of tomato mutations within SlPSKR1 identified following TILLING strategy. The genomic regions of SlPSKR1 which have been targeted in the TILLING method are indicated by arrows Target 1 and Target 2. The six mutations identified with the TILLING approach are the following: pskr1.1 A88 T, pskr1.2 T119 C, pskr1.3 G502 A, pskr1.4 G856 A, pskr1.5 G2285 A and pskr1.6 G1978 A. The drawing also represents protein domains as bottom arrows indicating the signal peptide (SP), the leucine-rich repat domain (LRR), the transmembrane domain (TM), and the kinase domain. Primer attachment sites for TILLING are indicated in capital letters.

### DETAILED DESCRIPTION OF THE INVENTION

The present invention provides novel and efficient methods for producing plants resistant to pathogens, having defective PSK and/or PSK receptor functions.

Surprisingly, the inventors have now discovered that PSKs act as negative regulators of plant resistance to plant pathogens, i.e., their inhibition increases resistance by reducing susceptibility. To our knowledge, this is the first example of a negative regulation of resistance in plants by growth factors. The PSK signaling pathway thus represents a novel and highly valuable target for producing plants of interest with increased resistance to pathogens. The inventors have further demonstrated that plants having defective PSK and/or PSK receptor functions have reduced susceptibility to different types of pathogens, such as oomycete, nematode and bacterial pathogens, showing the broad application of this invention.

The present disclosure will be best understood by reference to the following definitions:

### Definitions

As used therein, the term “PSK peptide” designates a sulfated phytosulfokine peptide acting as a negative regulator of plant resistance. Such a PSK peptide preferably comprises the amino acid sequence of H-Tyr(SO3H)-Ile-Tyr(SO3H)-Thr-OH (SEQ ID NO: 1) or the amino acid sequence of H-Tyr(SO3H)-Ile-Tyr(SO3H)-Thr-Gln-OH (SEQ ID NO: 2), or any natural variant thereof (e.g., variants present in other plants or which result from polymorphism). Preferably, a PSK peptide contains at least 4 amino acids. More preferably, a PSK peptide contains at least 5 amino acids. Typically, a PSK peptide contains at least two sulfated amino acid residues, which are preferably tyrosine residues. The term PSK peptide also designates any precursor or immature form of the peptide, such as for example PSK preproteins comprising amino acid sequences of SEQ ID NO: 3, 4, 5, 6 or 7. Specific examples of PSK precursors include Populus trichocarpa PSK precursors comprising a sequence selected from SEQ ID NO: 8-13, Oryza sativa PSK precursors comprising a sequence selected from SEQ ID NO: 14-19, 95, 97, 99, 101, 103, Vitis vinifera PSK precursors comprising a sequence selected from SEQ ID NO: 20-24 and Solanum lycopersicum precursors comprising a sequence selected from SEQ ID NO: 69, 71, 73 or 75.

Within the context of the present invention, the term “PSK gene” designates any nucleic acid that codes for a PSK peptide (or its precursor). The term “PSK gene” includes PSK DNA (e.g., genomic DNA) and PSK RNA (e.g., mRNA), as applicable. In particular, a “PSK gene” includes any nucleic acid encoding a phytosulfokine peptide or a natural variant of such a peptide, as defined above. Examples of PSK genes include the PSK genomic DNA or RNA of Arabidopsis thaliana, Solanum lycopersicum (Lycopersicon esculentum), Oryza sativa, Zea mays, Sorghum bicolor, Triticum aestivum, Asparagus officinalis, Brassica napus, Beta vulgaris, Solanum tuberosum, Glycine max, Vitis vinifera and Daucus carota. Specific example of a PSK gene comprises the nucleic acid sequence of SEQ ID NO: 25-29, 86-90 (Arabidopsis thaliana), SEQ ID NO: 68, 70, 72 or 74 (Solanum lycopersicum), SEQ ID NO: 94, 96, 98, 100, 102, 104, 105, (Oryza sativa).

Further examples of PSK genes or peptides are listed below:

Rice (Oryza sativa)

GenBank: BAF11381.2, 0s03g0232400

NCBI Reference Sequence: NP_{—}001050886.1, Swiss-Prot: Q9FRF9.1 Q9FRF9, PSK3

GenBank: AAG46077.1

GenBank: BAF12800.1

GenBank: EEC75912.1, hypothetical protein OsI_{—}12987

GENE ID: 4333708 0s03g0675600

GenBank: ABF98161.1, Phytosulfokines 3 precursor, putative

GenBank: EAZ28113.1, hypothetical protein OsJ_{—}12080

Maize (Zea mays)

GenBank: ACG49207.1, PSK4

GenBank: DAA00297.1, PSK

NCBI Reference Sequence: NP_{—}001105796.1, PSK1

GenBank: ACG23972.1, PSK

GenBank: ACG41544.1, phytosulfokine precursor protein

GenBank: ACG27399.1, phytosulfokine precursor protein

Sorghum (Sorghum bicolor)

GENE ID: 8085257 SORBIDRAFT_{—}01g042120

GENE ID: 8084300 SORBIDRAFT_{—}02g001950

GenBank: EES08686.1 SORBIDRAFT_{—}05g021760

Wheat (Triticum aestivum)

GenBank: DAA00296.1, putative phytosulfokine peptide precursor

GenBank: ABG66637.1, phytosulfokine-alpha 2 precursor

GenBank: ABG66638.1, phytosulfokine-alpha 2 precursor

Wild Asparagus (Asparagus officinalis)

Swiss-Prot: Q9FS10, PSK

GenBank: BAB20706.1, preprophytosulfokine

Rapeseed (Brassica napus)

GenBank: DAA00277.1, putative phytosulfokine peptide precursor

Beet (Beta vulgaris)

Swiss-Prot: CAK22422.1, phytosulfokine-alpha peptide precursor

Tomato (Solanum lycopersicum)

GenBank: DAA00287.1, PSK4

Potato (Solanum tuberosum)

GenBank: DAA00294.1, PSK

GenBank: DAA00293.1, PSK

Soybean (Glycine max)

GenBank: ACU23402.1, phytosulfokine peptide precursor

GenBank: DAA00280.1, putative phytosulfokine peptide precursor

GenBank: DAA00283.1, putative phytosulfokine peptide precursor

GenBank: DAA00282.1, putative phytosulfokine peptide precursor

GenBank: DAA00279.1, putative phytosulfokine peptide precursor

Grape (Vitis vinifera)

GenBank: CB138497.3, PSK

GenBank: CAN65538.1 and CBI25131.3, PSKs

GenBank: CBI19372.1, PSK

GenBank: CBI30250.3, unnamed protein product

GenBank: CBI17083.3

GenBank: CAN62427.1, hypothetical protein

Banana (Musa acuminata)

GenBank: ABF70025.1,phytosulfokine family protein

Zinnia (Zinnia violacea)

Swiss-Prot: Q8H0B9, preprophytosulfokine

Tree cotton (Gossypium arboreum)

GenBank: DAA00278.1, putative phytosulfokine peptide precursor

Poplar (Populus trichocarpa)

NCBI Reference Sequence: XP_{—}002320667.1, PSK

GenBank: EEE98982.1, PSK

NCBI Reference Sequence: XP_{—}002320021.1, PSK

NCBI Reference Sequence: XP_{—}002301142.1, PSK

GenBank: EEE87877.1

Pine tree (Pinus taeda)

GenBank: DAA00289.1, PSK

Douglas fir (Pseudotsuga menziesii)

GenBank: ACH59688.1

GenBank: ACH59689.1

GenBank: ACH59690.1

GenBank: ACH59691.1

GenBank: ACH59692.1

GenBank: ACH59693.1

GenBank: ACH59694.1

GenBank: ACH59695.1

GenBank: ACH59696.1

GenBank: ACH59697.1

GenBank: ACH59698.1

GenBank: ACH59699.1

GenBank: ACH59701.1

GenBank: ACH59702.1

GenBank: ACH59703.1

GenBank: ACH59704.1

GenBank: ACH59705.1

GenBank: ACH59706.1

GenBank: ACH59707.1

GenBank: ACH59708.1

GenBank: ACH59709.1

As used therein, the term “PSKR” or “PSK receptor” designates a receptor of a PSK peptide. Typically, a PSKR has an extracellular domain binding the PSK peptide as defined above, and an intracellular signaling domain having a kinase activity. The PSKR has been isolated and cloned from various species, including Arabidopsis thaliana, Solanum lycopersicum, Daucus carota, Oryza sativa, and Vitis vinifera. Illustrative sequences of a PSKR are provided as SEQ ID NO: 30, 31 (Arabidopsis thaliana), SEQ ID NO: 32 (Daucus carota), SEQ ID NO: 33 (Vitis vinifera), SEQ ID NO: 111, 113 (Populus trichocarpa), SEQ ID NO: 34, 107, 109 (Oryza sativa) and SEQ ID NO: 35, 114 (Solanum lycopersicum). The preferred PSKR according to the invention is PSKR1 receptor.

A “PSKR gene” designates any nucleic acid that codes for a PSKR receptor. In particular, a “PSKR gene” may be any DNA or RNA encoding a receptor of the phytosulfokine peptide, as applicable. Specific examples of PSKR gene include a nucleic acid comprising the sequence of SEQ ID NO: 36 or 37, which encode the amino acid sequences of PSKR1 or PSKR2 of Arabidopsis thaliana. In another embodiment, “PSKR gene” codes for any natural variant or homolog of a PSKR1 or PSKR2 protein. Examples of PSKR gene include the PSKR gene or RNA of Solanum lycopersicum, Daucus carota, Vitis vinifera. Illustrative sequences are provided as SEQ ID NO: 38, 39, 40, 67, 91, 92, 93, 108, 109, 110 or 112.

Within the context of the present invention, the term “pathogens” designates all pathogens of plants in general. More preferably the pathogens are fungal, oomycete, nematode or bacterial pathogens. In a particular embodiment, fungal pathogens are cereal fungal pathogens. Examples of such pathogens include, without limitation, Magnaporthe, Puccinia, Aspergillus, Ustilago, Septoria, Erisyphe, Rhizoctonia and Fusarium species.

In a more preferred embodiment, the pathogens are biotrophic or hemi-biotrophic oomycete pathogens selected from the genera of Phytophthora, Peronospora, Hyaloperonospora, and Plasmopara. The most preferred oomycete pathogens are Hyaloperonospora arabidopsidis, Phytophthora parasitica, Phytophthora infestans, Phytophthora capsici and Plasmopara viticola.

In another preferred embodiment, the pathogens are nematode pathogens. The most preferred nematode pathogens are Meloidogyne spp. (M. incognita, M. javanica, M. arenaria, M. hapla, M. graminicola), Globodera spp. and Heterodera spp.

In another preferred embodiment, the pathogens are bacterial pathogens. The most preferred bacterial pathogen is Ralstonia solanacearum.

Different embodiments of the present invention will now be further described in more details. Each embodiment so defined may be combined with any other embodiment or embodiments unless otherwise indicated. In particular, any feature indicated as being preferred or advantageous may be combined with any other feature or features indicated as being preferred or advantageous.

### PSK- or PSKR-defective Plants

As previously described, the present invention is based on the finding that PSK and PSKR genes are negative regulators of plant resistance to plant pathogens. The inventors have demonstrated that the inactivation of the PSK or PSKR gene(s) increases plant resistance to plant pathogens.

The present invention thus relates to methods for increasing pathogen resistance in plants based on a regulation of PSK pathways. The present invention also relates to methods of protecting a plant against pathogens by decreasing or suppressing PSK function in said plant.

The invention also relates to plants or plant cells having a defective PSK function.

The invention also relates to constructs (e.g., nucleic acids, vectors, cells, etc) suitable for production of such plants and cells, as well as to methods for producing plant resistant regulators.

According to a first embodiment, the invention relates to a plant or a plant cell comprising a defective PSK function. The term “PSK function” indicates any activity mediated by a PSK peptide or receptor in a plant cell. The PSK function may be effected by the PSK gene expression or the PSK peptide activity as well as the PSKR gene expression or the PSKR receptor activity.

Within the context of this invention, the terms “defective”, “inactivated” or “inactivation”, in relation to PSK function, indicate a reduction in the level of active PSK peptide or active PSKR receptor present in the cell or plant. Such a reduction is typically of about 20%, more preferably 30%, as compared to a wild-type plant. Reduction may be more substantial (e.g., above 50%, 60%, 70%, 80% or more), or complete (i.e., knock-out plants).

Inactivation of PSK or its receptor may be carried out by techniques known per se in the art such as, without limitation, by genetic means, enzymatic techniques, chemical methods, or combinations thereof. Inactivation may be conducted at the level of DNA, mRNA or protein, and inhibit the expression (e.g., transcription or translation) or the activity of PSK or PSKR.

Preferred inactivation methods affect expression and lead to the absence of production of a functional PSK peptide and/or PSKR receptor in the cells. It should be noted that the inhibition of PSK or PSKR may be transient or permanent.

In a first embodiment, defective PSK or PSKR is obtained by deletion, mutation, insertion and/or substitution of one or more nucleotides in one or more PSK or PSKR gene(s). In a preferred embodiment, all the PSK genes are inactivated in the plant of interest. This may be performed by techniques known per se in the art, such as e.g., site-specific mutagenesis, ethyl methanesulfonate (EMS) mutagenesis, targeting induced local lesions in genomes (TILLING), EcoTILLING, homologous recombination, conjugation, etc.

The TILLING approach according to the invention aims to identify SNPs (single nucleotide polymorphisms) and/or insertions and/or deletions in a PSK or PSKR gene from a mutagenized population. It can provide an allelic series of silent, missense, nonsense, and splice site mutations to examine the effect of various mutations in a gene. EcoTILLING is a variant of TILLING, which examines natural genetic variation in populations.

Another particular approach is gene inactivation by insertion of a foreign sequence, e.g., through transposon mutagenesis using mobile genetic elements called transposons, which may be of natural or artificial origin.

In the most preferred embodiment, the defective PSK or PSKR is obtained by knock-out techniques, e.g., deletion of all or a portion of the gene, the deleted portion having a size sufficient to prevent expression of a functional protein from the gene. The deleted portion preferably comprises at least 50 consecutive nucleotides of the gene. In a particular embodiment, the deleted gene or portion is replaced in the genome by an inserted foreign nucleic acid.

According to another preferred embodiment, the defective PSK or PSKR is obtained by gene silencing using RNA interference, ribozyme or antisense technologies. In a particular embodiment, an inhibitory nucleic acid molecule which is used for gene silencing comprises a sequence that is complementary to a sequence common to several PSK or PSKR genes or RNAs. Preferably, such an inhibitory nucleic acid molecule comprises a sequence that is complementary to a sequence present in all PSK genes or RNAs or PSKR genes or RNAs of a same species, e.g., Arabidopsis thaliana, Solanum lycopersicum, Oryza sativa, Zea mays, Sorghum bicolor, Triticum aestivum, Asparagus officinalis, Brassica napus, Beta vulgaris, Solanum tuberosum, Glycine max, Vitis vinifera and/or Daucus carota.

PSK or PSKR synthesis in a plant may also be reduced by mutating or silencing genes involved in the PSK or PSKR biosynthesis pathway, e.g. those encoding sulfotransferases (SOTs) required for sulfation of the PSK tyrosine residues. Alternatively, PSK or PSKR synthesis and/or activity may also be manipulated by (over)expressing negative regulators of PSK or PSKR, such as transcription factors or second messengers. In another embodiment, a mutant allele of a gene involved in PSK or PSKR synthesis may be (over)expressed in a plant.

PSK or PSKR inactivation may also be performed transiently, e.g., by applying (e.g., spraying) an exogenous agent to the plant, for example molecules that inhibit PSK or PSKR activity.

Preferred inactivation is a permanent inactivation produced by destruction of the integrity of the PSK or PSKR genes, e.g., by deletion of a fragment (e.g., at least 50 consecutive bp) of the gene sequence and/or by insertion of a foreign sequence. As illustrated in the examples, psk or pskr knock-out plants with a defective PSK or PSKR gene are still viable, show no aberrant developmental phenotype, and exhibit increased resistance to plant pathogens.

In a specific embodiment, more than one PSK or PSKR gene(s) are rendered defective by knock-out techniques.

In another embodiment, defective PSK function is obtained at the level of the PSK peptide. For example, the PSK peptide may be inactivated by exposing the plant to, or by expressing in the plant cells an antibody directed against the PSK peptide (e.g., anti-sulfotyro sine monoclonal antibody).

The PSK peptide may also be inactivated by exposing the plant to, or by overexpressing PSKR containing an extracellular binding domain but devoid of the intracellular signaling domain.

Alternatively, defective PSK function is obtained by alteration of the PSKR receptor activity. More specifically, the PSKR receptor may be inactivated by antagonists of the PSKR receptor. In a particular embodiment, such antagonists bind to the residues of Glu503-Lys517 of the PSKR receptor.

Thus, the PSK function in plant resistance may be controlled at the level of PSK genomic DNA, PSK mRNA, PSK peptide, PSKR genomic DNA, PSKR mRNA, or PSKR receptor activity.

In a variant, the invention relates to a plant with increased resistance to plant pathogens, wherein said plant comprises an inactivated PSK gene, more specifically an inactivated PSK genomic DNA. The defective PSK gene is preferably selected from PSK 1, PSK 2, PSK 3, PSK 4 and PSK 5. In another preferred embodiment, all the PSK genes present in the plant are defective, for example all of PSK1-5 genes.

In another variant, the invention relates to a plant with increased resistance to plant pathogens, wherein said plant comprises an inactivated PSK peptide.

In another variant, the invention relates to a plant with increased resistance to plant pathogens, wherein said increased resistance is due to inactivation of a PSKR genomic DNA. The defective PSKR gene may be the ortholog of the Arabidopsis PSKR1 gene.

In another variant, the invention relates to a plant with increased resistance to plant pathogens, wherein said increased resistance is due to inactivation of a PSK or PSKR mRNA.

In another embodiment, the invention relates to transgenic plants or plant cells which have been engineered to be (more) resistant to plant pathogens by inactivation of PSK function. In a particular embodiment, the modified plant is a loss-of-function psk or pskr mutant plant, with increased resistance to plant pathogens.

The invention also relates to seeds of plants of the invention, as well as to plants, or descendents of plants grown or otherwise derived from said seeds, said plants having an increased resistance to pathogens.

The invention also relates to vegetal material of a plant of the invention, such as roots, leaves, flowers, callus, etc.

The invention also provides a method for producing plants having increased resistance to pathogens, wherein the method comprises the following steps:
(a) inactivation of PSK and/or PSKR gene(s) in a plant cell;
(b) optionally, selection of plant cells of step (a) with defective PSK and/or PSKR gene(s);
(c) regeneration of plants from cells of step (a) or (b); and
(d) optionally, selection of a plant with increased resistance to pathogens, said plant with increased resistance to pathogens having defective PSK or PSKR gene(s).

Inactivation of the PSK and/or PSKR gene can be done as disclosed above. Genetic alteration in the PSK or PSKR gene may also be performed by transformation using the Ti plasmid and Agrobacterium infection method, according to the protocol described e.g., by Toki et al (2006). In a preferred method, inactivation is caused by PSK or PSKR gene destruction using e.g., knock-out techniques.

Selection of plant cells having a defective PSK and/or PSKR gene can be made by techniques known per se to the skilled person (e.g., PCR, hybridization, use of a selectable marker gene, protein dosing, western blot, etc.).

Plant generation from the modified cells can be obtained using methods known per se to the skilled worker. In particular, it is possible to induce, from callus cultures or other undifferentiated cell biomasses, the formation of shoots and roots. The plantlets thus obtained can be planted out and used for cultivation. Methods for regenerating plants from cells are described, for example, by Fennell et al. (1992) Plant Cell Rep. 11: 567-570; Stoeger et al (1995) Plant Cell Rep. 14: 273-278.

The resulting plants can be bred and hybridized according to techniques known in the art. Preferably, two or more generations should be grown in order to ensure that the genotype or phenotype is stable and hereditary.

Selection of plants having an increased resistance to a pathogen can be done by applying the pathogen to the plant, determining resistance and comparing to a wt plant.

Within the context of this invention, the term “increased resistance” to pathogen means a resistance superior to that of a control plant such as a wild type plant, to which the method of the invention has not been applied. The “increased resistance” also designates a reduced, weakened or prevented manifestation of the disease symptoms provoked by a pathogen. The disease symptoms preferably comprise symptoms which directly or indirectly lead to an adverse effect on the quality of the plant, the quantity of the yield, its use for feeding, sowing, growing, harvesting, etc. Such symptoms include for example infection and lesion of a plant or of a part thereof (e.g., different tissues, leaves, flowers, fruits, seeds, roots, shoots), development of pustules and spore beds on the surface of the infected tissue, maceration of the tissue, accumulation of mycotoxins, necroses of the tissue, sporulating lesions of the tissue, colored spots, etc. Preferably, according to the invention, the disease symptoms are reduced by at least 5% or 10% or 15%, more preferably by at least 20% or 30% or 40%, particularly preferably by 50% or 60%, most preferably by 70% or 80% or 90% or more, in comparison with the control plant.

The term “increased resistance” of a plant to pathogens also designates a reduced susceptibility of the plant towards infection with plant pathogens or lack of such susceptibility. The inventors have demonstrated, for the first time, a correlation between expression of PSK or PSKR genes and susceptibility towards infection. As shown in the experimental part, infection of plants with oomycete pathogens, triggers transcriptional activation of PSK and PSKR1 genes. Furthermore, the inventors have shown that the overexpression of PSK genes and of PSKR1 promotes disease, whereas the knockout of

PSK3 and of PSKR1 increases resistance. The inventors have therefore proposed that the PSK signaling increases susceptibility of plants to infection and favors the development of the disease. Thus, in a preferred embodiment, the resistance of PSK- or PSKR-defective plants to plant pathogens is due to a loss of susceptibility of these plants to pathogens.

Preferred plants or cells of the invention should be homozygous with respect to PSK or PSKR gene inactivation, i.e., both PSK or PSKR alleles are inactive.

In the most preferred embodiment, the method of the invention is used to produce dicot or monocot plants having a defective PSK or PSKR gene with increased resistance to oomycete, nematode and/or bacterial pathogens. Examples of such plants and their capacity to resist pathogens are disclosed in the experimental section.

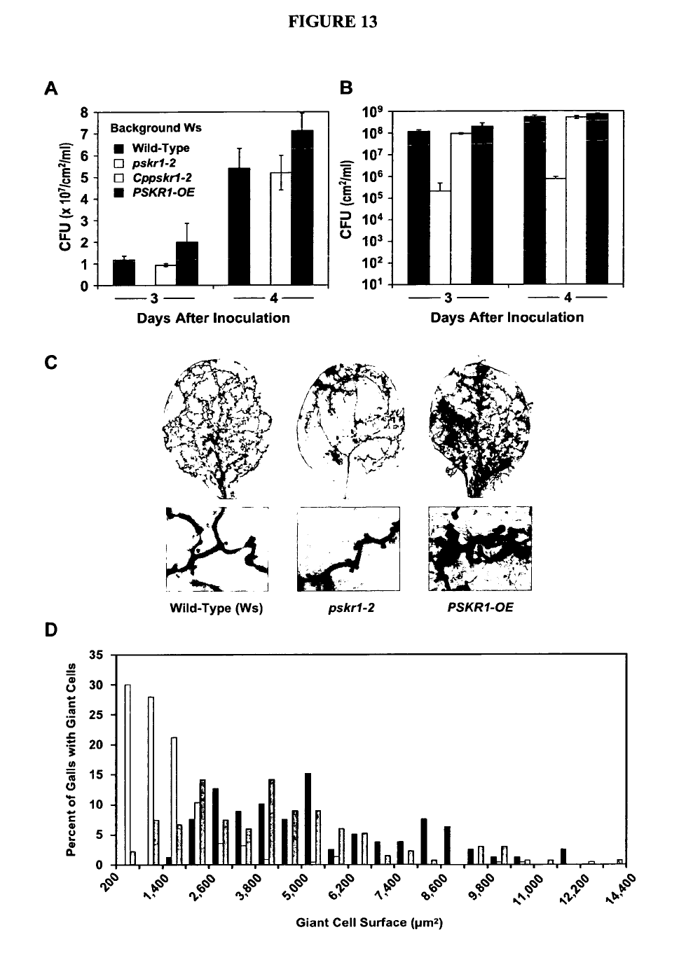

A particular object of the invention relates to a Solanaceae plant, preferably a tomato plant, wherein the cells of said plant lack all or part of a PSK or PSKR1 gene and are defective for PSK function. Such plants exhibit increased resistance to pathogens such as fungus, oomycetes, nematodes or bacterial pathogens. In a preferred embodiment, the invention relates to a tomato plant wherein the cells of said plant lack all or part of the PSKR1 gene. A preferred plant lacks at least a portion (i.e., more than 50 consecutive nucleotides) of the gene within target1 or target2 as disclosed FIG. 13. Even more preferably, the deleted portion encompasses at least one of the following nucleotides: A88, T119, G502, G856, G2285 and G1978.

Another particular object of the invention relates to a Solanaceae plant, preferably a tomato plant, wherein the cells of said plant have a mutated PSKR1 gene and are defective for PSK function. Such plants exhibit increased resistance to pathogens such as fungus, oomycetes, nematodes or bacterial pathogens. In a preferred embodiment, the mutation is present in target1 or target2 domains as disclosed FIG. 13. Even more preferably, the mutation is selected from pskr1.1 A88 T, pskr1.2 T119 C, pskr1.3 G502 A, pskr1.4 G856 A, pskr1.5 G2285 A and pskr1.6 G1978 A.

Another particular object of the invention relates to a Apiaceae plant, preferably a carrot plant, wherein the cells of said plant lack all or part of a PSK or PSKR1 gene and are defective for PSK function. Such plants exhibit increased resistance to pathogens such as fungus, oomycetes, nematodes or bacterial pathogens.

Another particular object of the invention relates to a Poaceae plant, preferably a wheat, rice, barley, oat, rye, sorghum or maize plant, wherein the cells of said plant lack all or part of a PSK or PSKR1 gene and are defective for PSK function. Such plants exhibit increased resistance to pathogens such as fungus, oomycetes, nematodes or bacterial pathogens.

### Screening of Plant Resistance Modulators

The invention also discloses novel methods of selecting or producing regulators of plant resistance, as well as tools and constructs for use in such methods.

In a particular aspect, the invention relates to a method for screening or identifying a molecule that modulates plant resistance, the method comprising testing whether a candidate compound modulates PSKR gene expression or activity. The test can be performed in a cell containing a reporter DNA construct cloned under control of PSKR promoter sequence, or in a cell expressing PSKR or PSKR fusion protein.

Preferably, such a method comprises the following steps:
providing a cell comprising a nucleic acid construct that comprises the sequence of a PSKR gene promoter operably linked to a reporter gene;
contacting the cell with a candidate molecule;
measuring the activity of PSKR promoter by monitoring of the expression of a marker protein encoded by the reporter gene in the cell; and
selecting a molecule that modulates the expression of the marker protein.

In another embodiment, the invention also relates to methods for screening or identifying a molecule that modulates the PSKR activity, comprising the following steps:
providing a cell comprising a reporter gene under the control of a transcription factor, and a fusion protein comprising a PSKR protein fused to the DNA binding domain of the transcription factor;
contacting said cell with another fusion protein comprising a candidate molecule fused to the transcriptional activation domain of the transcription factor;
measuring the activity of the PSKR by monitoring of the expression of a marker protein encoded by the reporter gene in the cell, said marker protein being expressed only if both fusion proteins are interacting;
selecting a molecule that induces the expression of the marker protein.

Preferred modulators are inhibitors of the expression of PSKR.

In a further embodiment, the invention also relates to the use of compounds that inhibit PSKR expression or activity for increasing resistance of plants to plant pathogens. Such compounds are typically identified using the above method of screening. The use of such compounds typically comprise exposing a plant to such compound, e.g., by spraying or in a mixture with water, thereby causing transient PSK inactivation, and transient increase in resistance to pathogens.

In this regard, the invention also relates to an antibody that specifically binds a PSK peptide or receptor, or a fragment or derivative of such antibody having essentially the same antigenic specificity. Such an antibody may be polyclonal or, more preferably, monoclonal. Examples of antibody fragments include Fab fragment, Fab′ fragment, CDR domains. Examples of derivatives include single chain antibodies, humanized antibodies, recombinant antibodies, etc. Such antibodies may be produced by techniques known per se in the art, such as immunization and isolation of polyclonal antibodies or, immunization, isolation of antibody-producing cells, selection and fusion thereof with e.g., myeloma cells, to produce hybrodima producing monoclonal antibodies. Fragments and derivatives thereof may be prepared using known techniques. An antibody specific for a PSK peptide or receptor is an antibody that binds such a peptide or receptor with a higher affinity than other peptides or receptors. Preferred specific antibodies essentially do not bind other peptides or receptors.

In another embodiment, the invention also relates to methods for identifying proteins, which interact with PSKR, which are required for functional PSKR signaling, and which might be additional targets for inactivation to increase resistance. Such screening methods are preferentially Y2H systems that allow identifying interaction partners of cytoplasmic and membrane-bound proteins, such as the split-ubiquitin system (Stagljar et al., 1998), and the mating-based split-ubiquitin system (Grefen et al., 2009). Proteins interacting with individual PSKR domains might also be identified with the classical GAL4 Y2H system that works in the yeast nucleus (Fields and Song, 1989).

Further aspects and advantages of the invention are provided in the following examples, which are given for purposes of illustration and not by way of limitation.

### EXAMPLES

### Materials and Methods

### Generation of Mutant and Transgenic Arabidopsis Lines for the Functional Analysis of Genes Encoding the Phytosulfokines PSK1, PSK2, PSK3, PSK4, PSK5 and Their Receptor PSKR1.

Several mutant and transgenic lines listed in Table 1 have been analyzed by the inventors.

TABLE 1
Mutant and transgenic Arabidopsis lines

| | | | | | Amplification | | |
|---|---|---|---|---|---|---|---|
| AGI | Gene | FST | Clone [symbol] ame | Line | attB1-attB2 | Vector | Ecotype |
| At2g02220 | PSKR1 | SAIL_245_H03.V1 | pskr1-1 | Mutant | | | Col N8846 |
| At2g02220 | PSKR1 | 407D02 | pskr1-2 | Mutant | | | Ws |
| At2g02220 | PSKR1 | 308B10 | pskr1-3 | Mutant | | | Col-0 |
| At2g02220 | PSKR1 | SALK-008585 | pskr1-4 | Mutant | | | Col-0 CS60000 |
| At2g02220 | PSKR1 | | Cppskr1-2 | pskr [symbol] -2 | 5472 bp | pHGW | Ws |
| | | | | complementation | | (Karimi et al, 2002) | |
| At2g02220 | PSKR1 | | p35s:PSKR1 | PSKR1 overexpression | 3060 bp | pH2GW7 | Ws |
| | | | | | | (Karimi et al, 2002) | |
| At2g02220 | PSKR1 | | p35s:PSKR1:GFP | PSKR1 overexpression | 3056 bp | pK7FWG2.0 | Ws |
| | | | | with C-terminal GFP | | (Karimi et al, 2002) | |
| At2g02220 | PSKR1 | | PSKR1pro:GFP:GUS | PSKR1 expression | 1795 bp | pKGWFS7 | Ws |
| At2g02220 | PSKR1 | | | analysis | | (Karimi et al, 2002) | |
| At1g13590 | PSK1 | SALK_036304 | psk1-1 | Mutant | | | Col-0 CS60000 |
| At2g22860 | PSK2 | | p35s:PSK2 | PSK2 overexpression | 294 bp | pK2GW7 | Ws |
| | | | | | | (Karimi et al, 2002) | |
| | | | PSK2pro:GFP:GUS | PSK2 expression | 1005 bp | pKGWFS7 | Ws |
| | | | | analysis | | (Karimi et al, 2002) | |
| | | | p35s:PSK [symbol] :GFP | PSK2 overexpression | 291 bp | pK7FWG2.0 | Col-0 |
| | | | | with C-terminal GFP | | (Karimi et al, 2002) | |
| | | | PSK2-RNAi | PSK2-RNAi | 291 bp | pH7GWTWG2(II) | Ws |
| | | | | | | (Karimi eta al, 2002) | |
| At3g44735 | PSK3 | SAIL_378_F03 | psk [symbol] -1 | Mutant | | | Col N8846 |
| At3g49780 | PSK4 | | p35s:PSK4 | PSK4 overexpression | 282 bp | pK2GW7 | Ws |
| | | | | | | (Karimi et al, 2002) | |
| At5g6 [symbol] 870 | PSK5 | SALK_043834 | psk5-1 | Mutant | | | Col-0 CS60000 |
| | PSKα | | p35S:spPSK4-pepPSK | PSKα overexpression | 135 bp | pK2GW7 | Ws |
| | | | | | | (Karimi et al, 2002) | |
| | PSKα | | p35S:spPSK4-pepPSK-HA | PSKα overexpression | 228 bp | pK2GW7 | Ws |
| | | | | with C-terminal HA [symbol] | | (Karimi et al, 2002) | |

[symbol]

indicates data missing or illegible when filed

For p35s:PSK2, a fragment of 294 by of entire coding sequence was amplified by PCR using the primers attB1 (5′-AAAAAGCAGGCTTCACCATGGCAAACGTCTCCGCTTTGC-3′; SEQ ID NO: 41) and attB2 (5′-AGAAAGCTGGGTGTCAAGGATGCTTCTTCTTCTGG-3′; SEQ ID NO: 42). The PCR fragment was inserted into the pDON207 donor vector and then in the plant expression vector pK2GW7 (Karimi et al., 2002) using Gateway technology (Invitrogen). The T-DNA from the resulting vector was transferred into the Ws wild-type by Agrobacterium-mediated transformation.

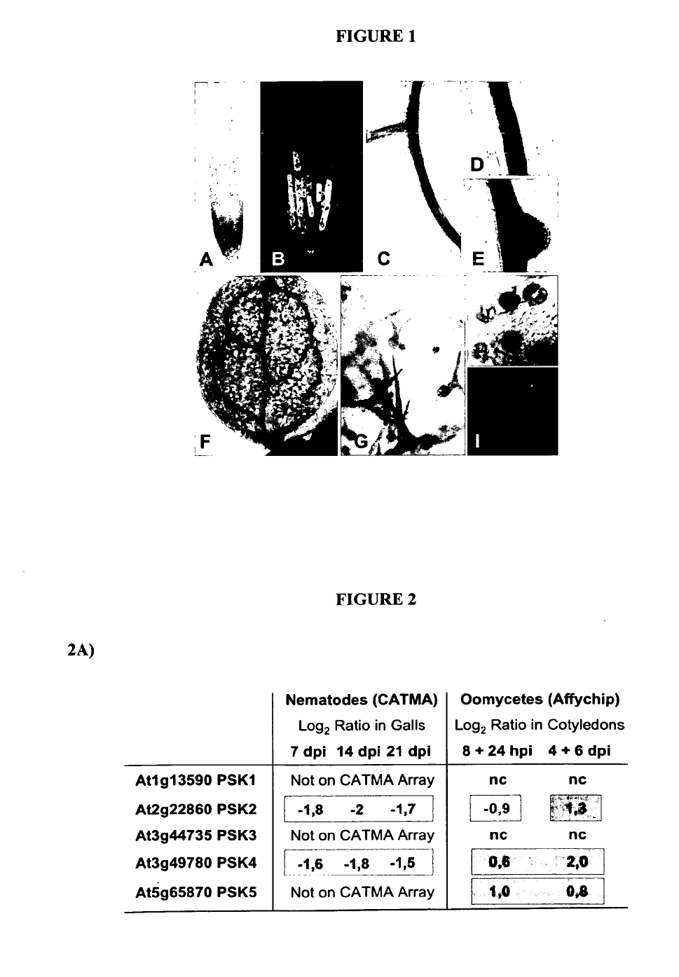

For PSK2pro:GFP: GUS fusion, a fragment of 1005 by upstream of the start codon was amplified by PCR using the primers attB1 5′-AAAAAGCAGGCTTCTGAAGTTTGGTGCATTAATTTA-3′; SEQ ID NO: 43) and attB2 (5′-AGAAAGCTGGGTGTTTTGTGATATTTTCTTTGAAG-3′; SEQ ID NO: 44). The PCR fragment was inserted into the pDON207 donor vector and then in the plant expression vector pKGWFS7 (Karimi et al., 2002) using Gateway technology (Invitrogen). The T-DNA from the resulting vector was transferred into the Ws wild-type by Agrobacterium-mediated transformation. Using the PSK2pro:GFP:GUS construction, the inventors have demonstrated that PSK2 gene is developmentally regulated (FIG. 1).

For p35s:PSK2:GFP fusion and PSK2-RNAi, a fragment of 291 by of entire coding sequence without stop codon was amplified by PCR using the primers attB1 (5′-AAAAAGCAGGCTTCACCATGGCAAACGTCTCCGCTTTGC-3′; SEQ ID NO: 45) and attB2 (5′-AGAAAGCTGGGTGAGGATGCTTCTTCTTCTGG-3′; SEQ ID NO: 46). The PCR fragment was inserted into the pDON207 donor vector and then in the plant expression vector pK7FWG2,0 (Karimi et al., 2002) for p35s:PSK2:GFP or pH7GWIWG2(II) (Karimi et al., 2002) for PSK2-RNAi using Gateway technology (Invitrogen). The T-DNAs from the resulting vectors were transferred into Col and Ws wild-types, respectively, by Agrobacterium-mediated transformation.

For p35s:PSK4, a fragment of 282 by of entire coding sequence was amplified by PCR using the primers attB1 (5′-AAAAAGCAGGCTTCACCATGGGTAAGTTCACAACCATTT-3′; SEQ ID NO: 47) and attB2 (5′-AGAAAGCTGGGTGTCCACCTCCGGATCAGGGCTTGTGATTCTGAGTA-3′; SEQ ID NO: 48). The PCR fragment was inserted into the pDON207 donor vector and then in the plant expression vector pK2GW7 (Karimi et al., 2002) using Gateway technology (Invitrogen). The T-DNA from the resulting vector was transferred into the Ws wild-type by Agrobacterium-mediated transformation.

The trangenic line spPSK4-pepPSK was generated to constitutively express a fusion between the PSK4 signal sequence for secretion and the PSKα minimal motif. A fragment of 113 by comprising the fusion was obtained by annealing the two primers, forPSK4PS-PSK (5′-AATTCATGGGTAAGTTCACAACCATTTTCATCATGGCTCTCCTTCTTTGCTCTA CGCTAACCTACGCAGAAGAGTTTCATACGGACTACATCTACACTCAGGACGT AA-3′; SEQ ID NO: 49) and revPSK4PS-PSK (5′-AGCTTTACGTCCTGAGTGTAGATGTAGTCCGTATGAAACTCTTCTGCGTAGGT TAGCGTAGAGCAAAGAAGGAGAGCCATGATGAAAATGGTTGTGAACTTACC CATG-3′; SEQ ID NO: 50). This fragment was ligated into EcoRI/HindIII- digested pBlueScript. A 135 by PCR fragment obtained from this vector as a template using the primers attB1 forPSK-B1 (5′-AAAAAGCAGGCTTCATGGGTAAGTTCACAACC-3′; SEQ ID NO: 51) and attB2 revPSKstop-B2 (5′-AGAAAGCTGGGTATCACTTTACGTCCTGAGTGTAG -3′; SEQ ID NO: 52) was then inserted into the pDON207 donor vector and then in the plant expression vector pK2GW7 (Karimi et al., 2002) using Gateway technology (Invitrogen). The T-DNA from the resulting vector was transferred into the Ws wild-type by Agrobacterium-mediated transformation.

The trangenic line spPSK4-pepPSK-HA was generated to constitutively express a fusion between the PSK4 signal sequence for secretion and the PSKα minimal motif harboring a C-terminal HA tag. A fragment of 113 by was obtained by annealing of the two primers, forPSK4PS-PSK (5′-AATTCATGGGTAAGTTCACAACCATTTTCATCATGGCTCTCCTTCTTTGCTCTA CGCTAACCTACGCAGAAGAGTTTCATACGGACTACATCTACACTCAGGACGT AA-3′; SEQ ID NO: 53) and revPSK4PS-PSK (5′-AGCTTTACGTCCTGAGTGTAGATGTAGTCCGTATGAAACTCTTCTGCGTAGGT TAGCGTAGAGCAAAGAAGGAGAGCCATGATGAAAATGGTTGTGAACTTACC CATG -3′; SEQ ID NO: 54). This fragment was ligated into EcoRI/HindIII- digested pBlueScript. For 3HA-tag insertion, a fragment of 111 by was amplified by PCR using the primers forHA-Hind (5-′GGTAAGCTTTACCCATACGATGTTCCTG-3′; SEQ ID NO: 55) and revHA-XhoI (5-′GAACTCGAGTCAAGCGTAATCTGGAACGTC-3′; SEQ ID NO: 56) on pNX32-Dest with following digestion by HindIII/XhoI. Digested 3HA-tag fragment was ligated into HindIII/XhoI—digested pBlueScript containing the fusion between the PSK4 signal sequence and the PSKα minimal sequence (without stop codon). A fragment of 228 by was the amplified by PCR using the primers attB1 forPSK-B1 (5′-AAAAAGCAGGCTTCATGGGTAAGTTCACAACC-3′; SEQ ID NO: 57) and attB2 revPSK-HAstop-B2 (⁵′-AGAAAGCTGGGTGTCAAGCGTAATCTGGAACG-3′; SEQ ID NO: 58). The PCR fragment was inserted into the pDON207 donor vector and then in the plant expression vector pK2GW7 (Karimi et al., 2002) using Gateway technology (Invitrogen). The T-DNA from the resulting vector was transferred into the Ws wild-type by Agrobacterium-mediated transformation.

For Cppskr1-2, a fragment of 5472 by including 1771 by upstream of the start codon (promoter and 5′UTR), 3027 by of entire coding sequence and 650 by of 3′ non coding sequence (3′UTR and terminator) was amplified by PCR using the primers attB1 (5′-AAAAAGCAGGCTTCATGGCAAGAAAATGTGAGAC-3′; SEQ ID NO: 59) and attB2 (5′-AGAAAGCTGGGTGGAACCATTATAGGAAGCGTACTAATC-3′; SEQ ID NO: 60). The PCR fragment was inserted into the pDON207 donor vector and then in the plant expression vector pHGW (Karimi et al., 2002) using Gateway technology (Invitrogen). The T-DNA from the resulting plant expression vector was transferred into the pskr1-2 mutant by Agrobacterium-mediated transformation.

For p35s:PSKR1 (PSKR1-OE), a fragment of 3060 by of the entire coding sequence was amplified by PCR using the primers attB1 (5′-AAAAAGCAGGCTGTTCTTGAAATGCGTGTTCATCG-3′; SEQ ID NO: 61) and attB2 (5′-AGAAAGCTGGGTCTAGACATCATCAAGCCAAGAGAC-3′; SEQ ID NO: 62). The PCR fragment was inserted into the pDON207 donor vector and then in the plant expression vector pH2GW7 (Karimi et al., 2002) using Gateway technology (Invitrogen). The T-DNA from the resulting vector was transferred into the Ws wild-type by Agrobacterium-mediated transformation.

For p35s:PSKR1:GFP fusion, a fragment of 3056 by of entire coding sequence without stop codon was amplified by PCR using the primers attB1 (5′-AAAAAGCAGGCTTTACCATGCGTGTTCATCGTTTT-3′; SEQ ID NO: 63) and attB2 (5′-AGAAAGCTGGGTAGACATCATCAAGCCAAGAGACT-3′; SEQ ID NO: 64). The PCR fragment was inserted into the pDON207 donor vector and then in the plant expression vector pK7FWG2.0 (Karimi et al., 2002) using Gateway technology (Invitrogen). The T-DNA from the resulting vector was transferred into the Ws wild-type by Agrobacterium-mediated transformation.

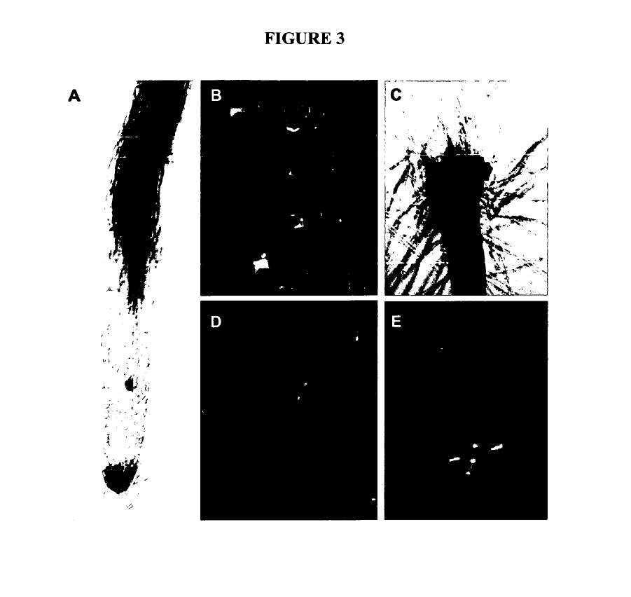

For PSKR1pro:GFP:GUS fusion, a fragment of 1795 by upstream of the start codon was amplified by PCR using the primers attB1 5′-AAAAAGCAGGCTTCATGGCAAGAAAATGTGAGAC-3′; SEQ ID NO: 65) and attB2 (5′-AGAAAGCTGGGTTTCAAGAACAGAGGAAGAAG-3′; SEQ ID NO: 66). The PCR fragment was inserted into the pDON207 donor vector and then in the plant expression vector pKGWFS7 (Karimi et al., 2002) using Gateway technology (Invitrogen). The T-DNA from the resulting vector was transferred into the Ws wild-type by Agrobacterium-mediated transformation. Using the PSKR1pro:GFP:GUS construction, the inventors have demonstrated that PSKR1 gene is developmentally regulated (FIG. 3).

### Example 1

### PSK Mutants are More Resistant to Infection By M. incognita and H. arabidopsidis

### I) PSK Expression During Plant Development:

Expression of the PSK2 gene during root and leaf development was analyzed through reporter gene activities in the transgenic line PSK2pro:GFP:GUS.

### Results:

As shown in FIG. 1, expression of the PSK2 gene is developmentally regulated. GUS activity (A) and GFP (B) revealing PSK2 promoter activation is detectable in the root tips (lateral root cap) but not in the elongation zone. In fully differentiated roots (C,D), PSK2 expression localizes to the vascular cylinder, and to the lateral root primordia (E). PSK2 expression in the shoots is localized in the vascular system of leaves and cotyledons (F), trichomes (G), and stomata (H,I).

### II) Gene Expression Analysis of Response to Pathogens Using Microarray:

Expression profiling of PSK genes during the compatible interaction with M. incognita and H. arabidopsidis was analyzed by microarray hybridizations. Samples were prepared from isolated galls and infected cotyledons at different time points after infection with M. incognita and H. arabidopsidis, respectively. Sample preparations, hybridizations on CATMA (M. incognita) and Affymetrix ATH1 (H. arabidopsidis) microarrays, and data analyses were performed as described (Jammes et al., 2005; Hok et al., 2011).

### Results:

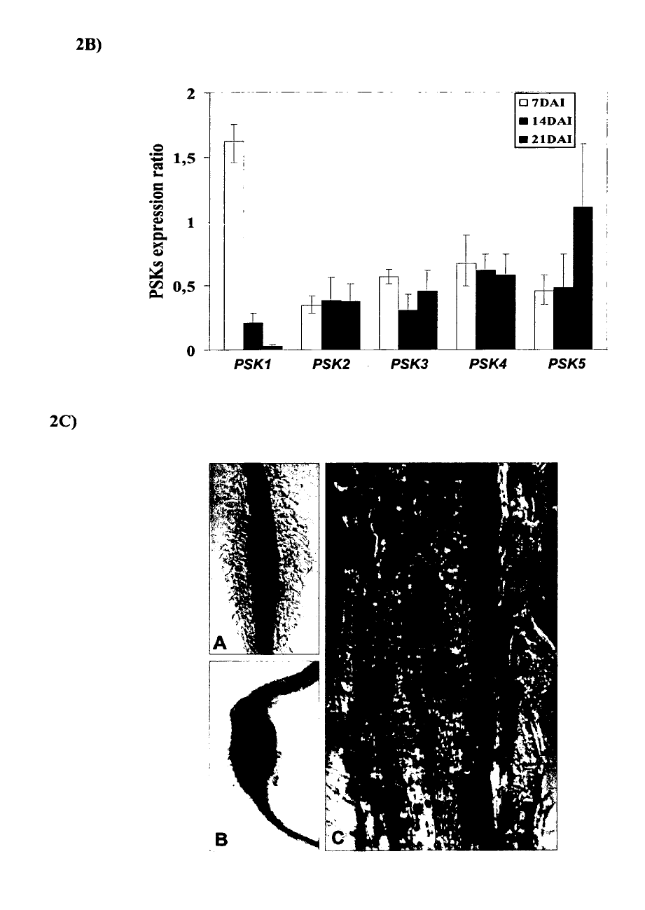

As shown in FIG. 2A, only genes encoding PSK2 and PSK4 are represented on CATMA arrays, and were downregulated at all stages of developing galls. The same genes were upregulated in infected cotyledons, particularly at late stages of downy mildew infection. Additionally, an upregulation of the gene encoding PSK5 is observed, whereas genes encoding PSK1 and PSK3 do not change (nc) expression intensities upon infection with H. arabidopsidis.

### III) Gene Expression Analysis of Response to Pathogens Using Real Time Quantitative RT-PCR

Relative PSK transcript accumulations in Arabidopsis galls were measured at 7 (white bars), 14 (grey bars), and 21 (black bars) days after nematode inoculation (DAI) by quantitative RT-PCR in comparison to uninfected roots. The PSK expression ratio was established with the 2^{-(ΔΔCt)} method, comparing the ΔCt for the gene of interest (Ct uninfected—Ct infected) with the ΔCt for the reference gene (Ct uninfected—Ct infected), where the gene of interest is one of the analyzed Arabidopsis PSK genes (PSK1-PSK5) and the reference gene is AtUBP22 (At5g10790). A ratio equaling 1 indicates that the PSK gene is not regulated by nematode infection. A ratio <−1 and >1 indicate gene repression and activation, respectively. Two biological replicates were performed. The results are shown in FIG. 2B.

### IV) Gene Expression Analysis of Response to Pathogens Using Reporter Gene Expression

PSK2 expression pattern was analyzed in galls of M. incognita-infected roots of the Arabidopsis PSK2pro:GFP:GUS reporter line as shown in FIG. 2C. Images A and B of FIG. 2C show a reduced GUS activity which is revealed in the center of galls forming at 5 (A) and 14 (B) days after inoculation. Image C of FIG. 2C shows projections of serial confocal optical in vivo sections show a downregulation of GFP accumulation representing PSK2 expression in giant cells.

### V) Quantitative Analysis for the Interaction Phenotype of PSK Knock-out Mutants

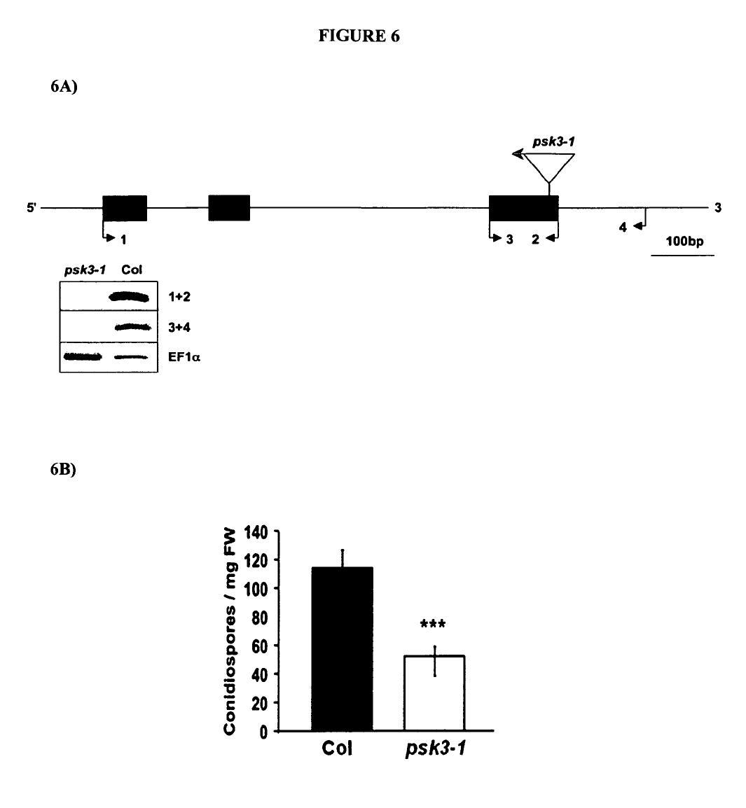

Quantitative analysis for the interaction phenotype of the psk3 knockout mutant (FIG. 6A) with H. arabidopsidis was carried out (FIG. 6B). Seeds from the different A. thaliana lines were sown on a soil/sand mixture, stratified for 3 days at 4° C., and then grown under a 12 h photoperiod in a growth chamber at 20° C. The H. arabidopsidis isolate, Emwa1 and Noco2 were transferred weekly onto the susceptible accession Ws-0 and Col-0, respectively, as described previously (Dangl et al., 1992). For infection, 10-day-old plants were spray-inoculated to saturation with a spore suspension of 40,000 spores/ml of the virulent isolate Noco2. Plants were kept in a growth cabinet at 16° C. for 6 d with a 12 h photoperiod. Sporulation was induced by spraying plants with water, and keeping them for 24 h under high humidity. Plantlets were collected 7 days post inoculation in 1 ml of water, vortexed, and the titer of liberated conidiospores was determined with a hemocytometer. Sporulation of H. arabidopsidis isolate Noco2 on cotyledons of the Arabidopsis psk3-1 mutant was reduced by >50%, when compared to wild-type plants (Col-0). Plantlets were collected 7 days post inoculation in 1 ml of water, vortexed, and the titer of liberated conidiospores was determined with a hemocytometer. For statistics, 20 samples at 10 plantlets were prepared for each line and analysis. The experiment was repeated 3 times with similar results. Statistically significant differences for values compared with the wild type were determined by Student's t-test (*** P<0.0001).

### Example 2

### Pskr1 Knock-out Mutants are Less Susceptible to H. arabidopsidis

Molecular analyses of 4 allelic Arabidopsis pskr1 knockout mutants have been conducted. The mutant lines pskr1-1 (SAIL_{—}245_H03), pskr1-2 (FLAG_{—}407D02), pskr1-3 (GABI_{—}308B10), and pskr1-4 (SALK-008585) were from the Syngenta Arabidopsis Insertion Library, from INRA (Versailles, France), from the Max-Planck-Institut (Cologne, Germany), and from the SALK Institute (LaJolla, USA), respectively. All lines are publicly available and were obtained from the Nottingham Arabidopsis Stock Center (pskr1-1, pskr1-3, and pskr1-4) and INRA Versailles (pskr1-2).

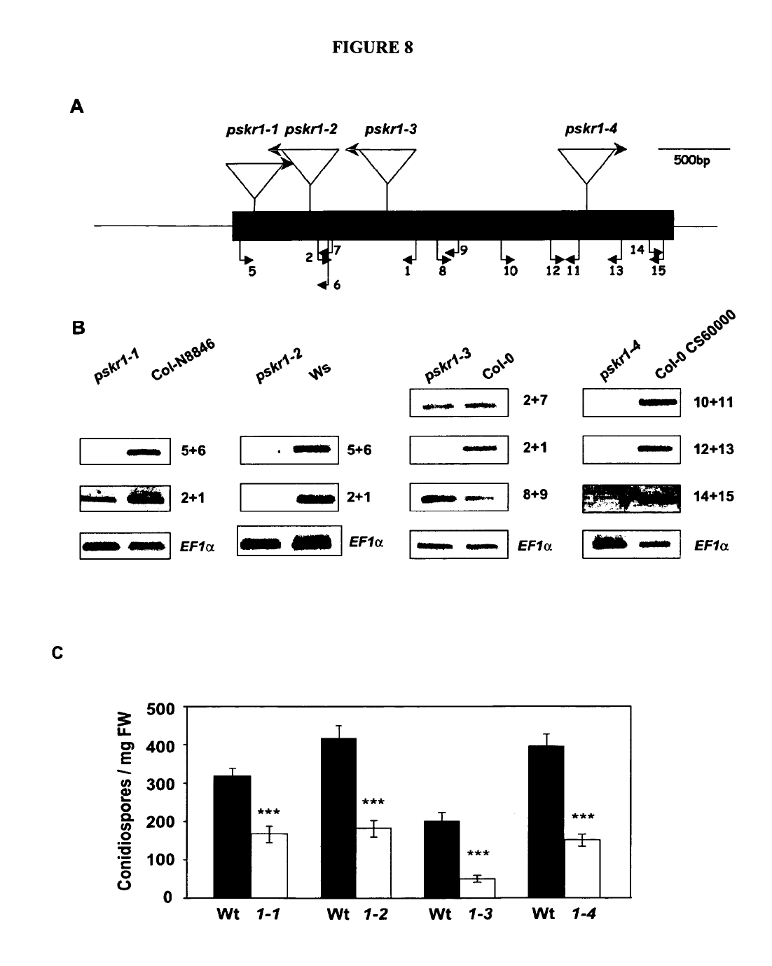

Primer attachment sites, and T-DNA insertion sites and orientations in the genome are indicated in FIG. 8A.

RT-PCR revealed PSKR1 transcripts in wild-type Arabidopsis (Col-N8846, Ws, Col-0, and Col-8 CS60000) as shown in FIG. 8B. Amplicons spanning the insertion sites were absent from all allelic mutants. Amplicons revealing transcripts with primers 3′ of the insertion sites most likely originate from transcriptional initiation within the T-DNA, as previously reported for other insertion lines (Chinchilla et al., 2007, Nature 448, 497-500). Amplification of transcripts from the constitutively expressed AtEF1α gene (At1g07930) showed that similar amounts of intact cDNAs were used for RT-PCR experiments.

For infection, 10-day-old plants were spray-inoculated to saturation with a spore suspension of 40,000 spores/ml of the virulent isolate (Emwa1 on the Ws wild-type and pskr1-2, Noco2 on the other wild-types and mutants). For statistical analysis of sporulation, 20 samples at 10 plantlets were prepared for each line and analysis. The bars represent mean values (±SD), and *** indicates significant differences between wild-type and mutant lines with P<0.0001, as determined by Student' s t-test. All experiments were repeated 3 times and gave similar results. 1-1, 1-2, 1-3, and 1-4 represent the mutants pskr1-1, pskr1-2, pskr1-3, and pskr1-4, respectively.

### Results:

As shown in FIG. 8C, all allelic pskr1 knock-out mutants exhibit an increased downy mildew resistance. Asexual reproduction, an indicator for disease provoked by the downy mildew oomycete pathogen, is reduced by >50%.

### Example 3

### Pskr1 Knock-out Mutants are Less Susceptible to M. incognita

Arabidopsis plants were infected in vitro 14 days after germination with 150 surface-sterilized freshly hatched M. incognita J2. Infected seedlings were kept at 20° C. with a 16-h photoperiod. During infection tests, egg mass counting was performed 60 DAI (days after inoculation) to allow nematodes to complete their life cycle. The nematode infects roots and initiates gall formation to a similar extent in pskr1 mutants and wild-type plants, as analyzed 10 days post inoculation (Dpi). A reduction in the amount of mature galls is observed in pskr1 mutants at 21 Dpi. The inhibition of nematode development in the absence of PSKR1 becomes most evident during the parthenogenetic production of egg masses, which are strongly reduced on pskr1 mutants at 75 Dpi.

### Results:

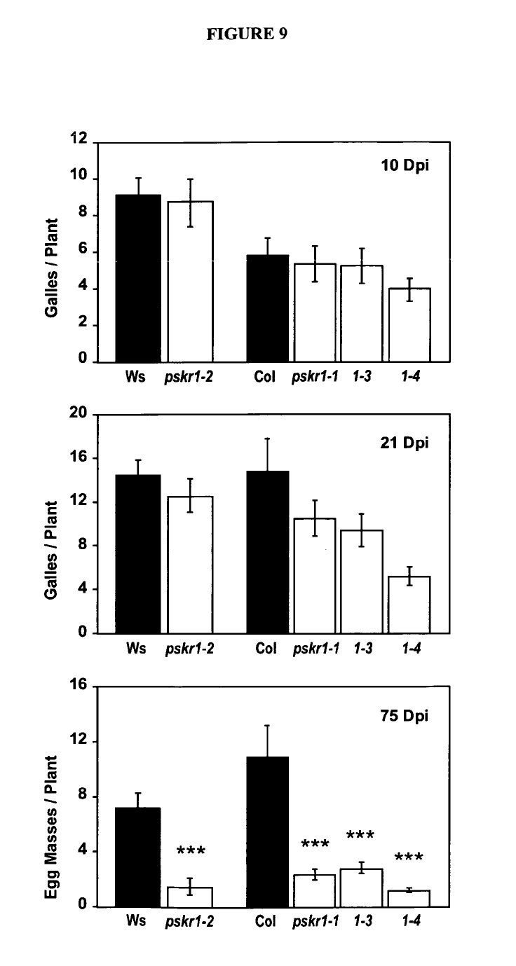

As shown in FIG. 9, allelic pskr1 mutants are less susceptible to M. incognita since root knot nematode reproduction is strongly inhibited in the absence of PSKR1. The production of galls and egg masses, which are indicators for disease provoked by the root knot nematode, is strongly reduced.

### Example 4

### The pskr1 Knock-out Mutants are Less Susceptible to Infection by R. Solanacearum

A thaliana seeds were sterilized for 20 min with a 12% sodium hypochlorite solution, washed several times with sterile water and sown on MS medium. Plantlets grown for 8 days at 20° C. in a growth chamber were then transferred to Jiffy pots (Jiffy France, Lyon, France) and grown for 3 weeks in short day conditions (10 h light at 500 μEs⁻¹m⁻²). Plants with a Ws and Col genetic background (mutant plants, complemented mutant plants, and plants overepressing PSKR1) were root-inoculated with the virulent bacterial isolates RD15 and GMI1000, respectively. Approximately 2 cm were cut from the bottom of the Jiffy pots and the exposed roots of the plants were immersed for 3 min in a suspension containing 10⁷ bacteria per ml. The plants were then transferred to a growth chamber with a day/night cycle of 8 h at 27° C., 120-140 μE m⁻¹s⁻² and 16 h at 26° C., respectively, keeping relative humidity at 75%. Disease symptoms on inoculated plants were scored at 3, 4, 5, 6, and 7 days post inoculation according to a disease index (DI) covering DI 0 (no wilt), and DI 1, DI 2, DI 3, and DI 4, representing 25%, 50%, 75%, and 100% of wilted leaves.

### Results:

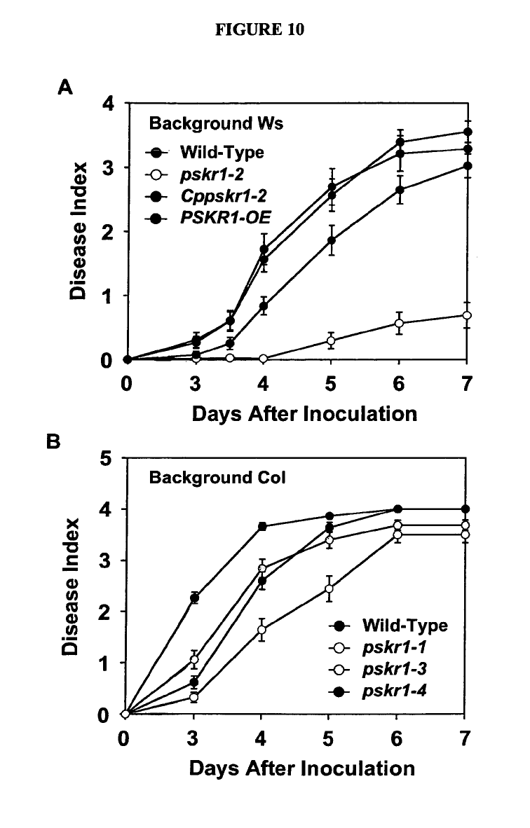

pskr1 knock-out mutants exhibit a reduced susceptibility to the bacterial pathogen Ralstonia solanacearum since the appearance of bacterial wilt symptoms was delayed in the absence of PSKR1 (FIG. 10). The observed enhanced resistance during the exponential bacterial growth phase between 3 and 5 days post inoculation was significant, with P<0.0001. The Col genetic background (FIG. 10B) of A. thaliana showed an overall higher susceptibility to R. solanacearum, and the effect of the pskr1 mutation is most pronounced in pskr1-2 in the Ws genetic background (FIG. 10A). Full susceptibility to R. solanacearum was restored through the introduction of a functional PSKR1 gene into the pskr1-2 genetic background (complemented line Cppskr1-2). An acceleration of disease at late time points of infection was observed in the line overexpressing PSKR1 under the control of the constitutive 35S promoter (overexpressing line PSKR1-OE).

### Example 5

### PSKR1 Gene Expression Pattern in Arabidopsis thaliana After Infection With the Downy Mildew Oomycete Pathogen, H. arabidopsidis

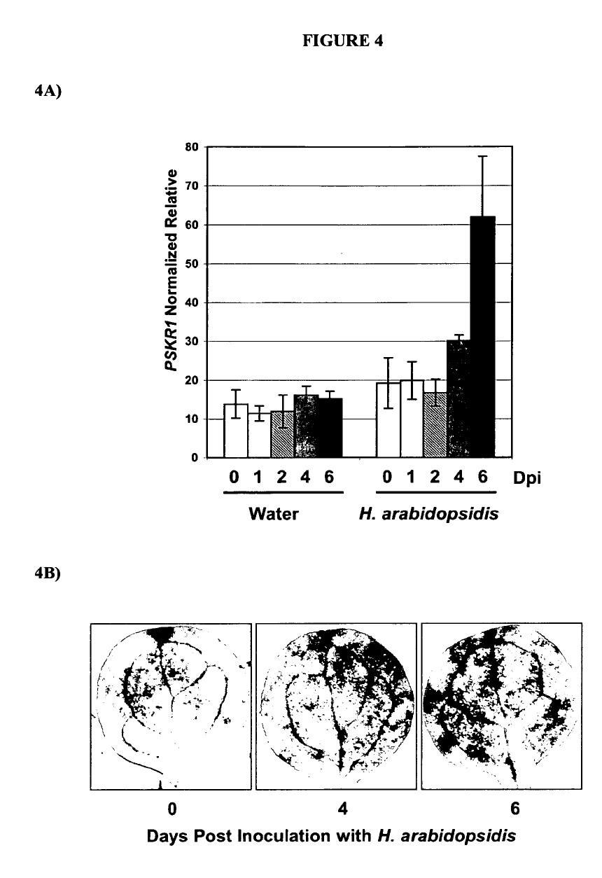

PSKR1 transcript abundance was analyzed by qRT-PCR at different time points after spray-treatment of Arabidopsis (ecotype Ws-0) cotyledons with water, or with conidiospore suspensions at 40,000 spores/ml of the downy mildew pathogen, H. arabidopsidis (Hpa) (see FIG. 4A). As shown in FIG. 4B, after infection, the expression of PSKR1 increases continuously and localizes to infected areas of the mesophyll.

### Example 6

### Infection with the Root-knot Nematode, M. incognita, Does Not Trigger Transcriptional Activation of the PSKR1 Gene, but Downregulates Expression in Giant Cells

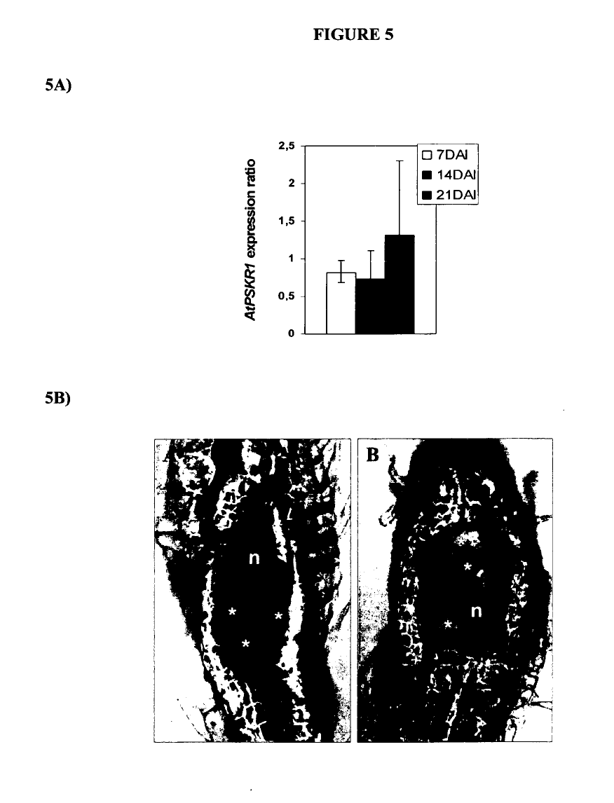

PSKR1 transcript abundance was first analyzed by qRT-PCR at 7, 14 and 21 days after root inoculation. Arabidopsis plants were infected in vitro 14 d after germination with 150 surface-sterilized freshly hatched M. incognita J2 larvae. Infected seedlings were kept at 20° C. with a 16-h photoperiod. Relative PSKR1 mRNA quantities were normalized with AtUBP22 (At5g10790) using Q-Base. The ratio equals 1 meaning that the PSKR1 gene is not regulated by nematode infection (FIG. 5A). The expression pattern of the GFP reporter gene under control of the PSKR1 promoter in galls was induced by M. incognita in Arabidopsis roots, 7 (A) and 21 (B) days after inoculation with 150 surface-sterilized freshly hatched M. incognita J2 larvae (FIG. 5B). Interestingly, PSKR1 expression appears being downregulated in giant cells induced by the nematode.

The inventors have hypothesized that PSKR is directly involved in giant cell ontogenesis or may have a role in the cells surrounding the giant cells (where PSKR is expressed) for their divisions or de novo formation of vascular elements. The surrounding cells should be also important to obtain functional feeding cells, specialized sinks that constitute the exclusive source of nutrients for the nematode until reproduction.

### Example 7

### Plants Over-expressing the PSK Gene are More Susceptible to H. arabidopsidis and M. incognita

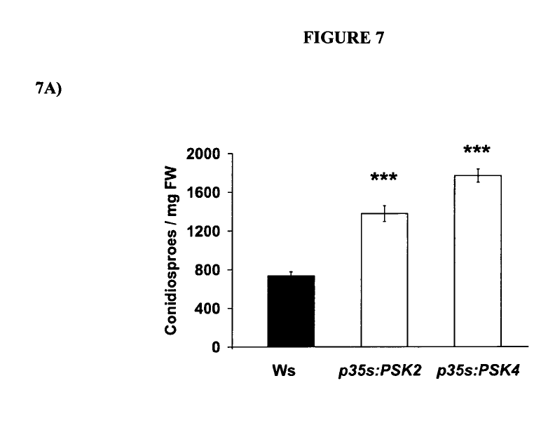

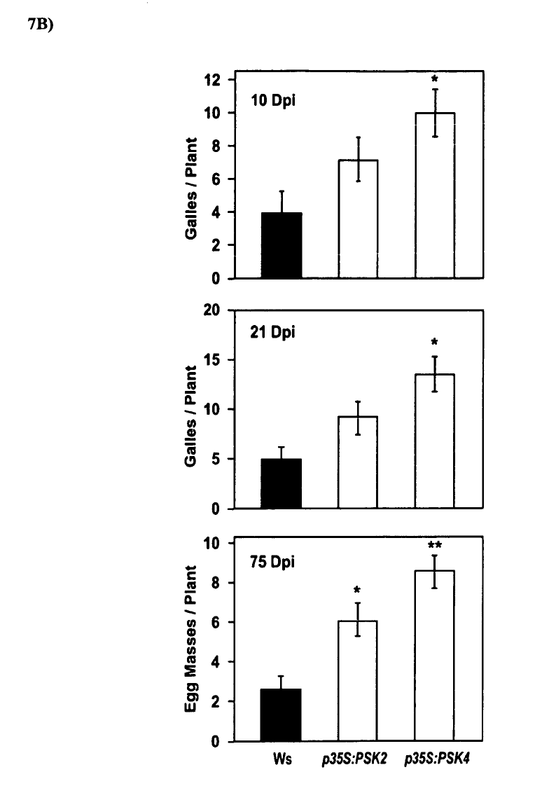

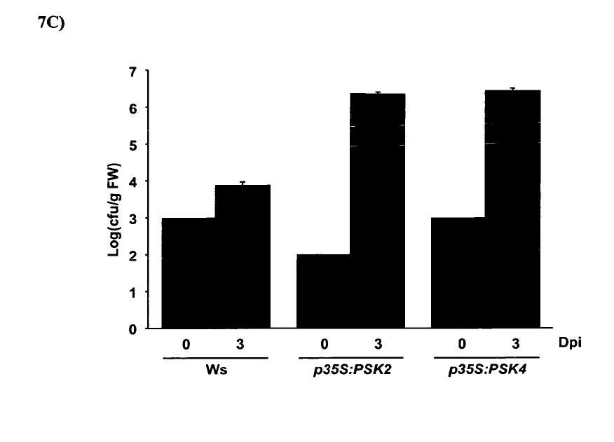

Quantitative analysis for the interaction phenotype with H. arabidopsidis of transgenic lines overproducing PSK2 and PSK4 was conducted. Sporulation of H. arabidopsidis isolate Emwa1 on cotyledons of the Arabidopsis PSK overexpressing lines is strongly increased, when compared to wild-type plants (Ws). For statistics, 20 samples at 10 plantlets were prepared for each line and analysis. The experiment was repeated 3 times with similar results. Statistically significant differences for values compared with the wild type were determined by Student's t-test (*** P<0.0001) as shown in FIG. 7A.

FIG. 7B shows that root knot nematode reproduction is significantly stimulated in transgenic lines constitutively overexpressing PSKs. Arabidopsis plants were infected in vitro 14 d after germination with 150 surface-sterilized freshly hatched M. incognita J2. Infected seedlings were kept at 20° C. with a 16-h photoperiod. During infection tests, egg mass counting was performed 75 Dpi (days post inoculation) to allow nematodes to complete their life cycle. The nematode infects roots and initiates gall formation, and develops mature galls to a stronger extent in PSK overexpressing plants than in wild-type plants, as analyzed 10 days and 21 Dpi, respectively. Statistically significant differences were determined by the Student's t test (* P<0.01, ** P<0.001, *** P<0.0001).

To determine the susceptibility of PSK overexpressing lines to R. solanacearum, bacterial growth curves were established. Four week-old plants were root-inoculated with a solution containing 10⁷ bacteria per ml of the virulent bacterial isolates RD15. The plants were then transferred to a growth chamber with a day/night cycle of 8 h at 27° C., 120-140 μE m⁻¹s⁻² and 16 h at 26° C., respectively, keeping relative humidity at 75%. For establishing bacterial internal growth curves, the aerial parts of three inoculated plants were weighed, sterilized with 250 ml of 70% ethanol for 3 min, rinsed three times in sterile water, and ground in a mortar after addition of sterile water (2.0 ml per g of fresh weight). Various dilutions of the ground material were then performed with sterile water and 3×40 μl of bacterial suspensions were spotted on petri plates containing solid SMSA medium (Elphinstone et al., 1996), and grown at 30° C. For each time point, triplicate assays were performed for each bacterial strain and A. thaliana accession.

### Results:

Plants overexpressing the PSK2 or PSK4 genes are more susceptible to H. arabidopsidis. A sexual reproduction, an indicator for disease provoked by the downy mildew oomycete pathogen, is significantly increased in both transgenic lines (FIG. 7A).
Transgenic plants overexpressing PSK2 or PSK4 genes are more susceptible to the nematode pathogen, M. incognita. Parthenogenetic production of egg masses at 75 Dpi is significantly enhanced in the transgenic lines, when compared to the wild-type (FIG. 7B).
Plants overexpressing the PSK2 or PSK4 genes are more susceptible to R. solanacearum. FIG. 7C shows that bacteria multiply faster in transgenic lines over-producing PSK2. Multiplication of R. solanacearum is strongly increased 3 Dpi, leading to a 100- to 1000-fold higher amount of bacteria in the infected PSK overexpressing lines, when compared to wild-type plants (FIG. 7C).

### Example 8

### Plants Over-expressing the PSKR Gene are More Susceptible to H. arabidopsidis

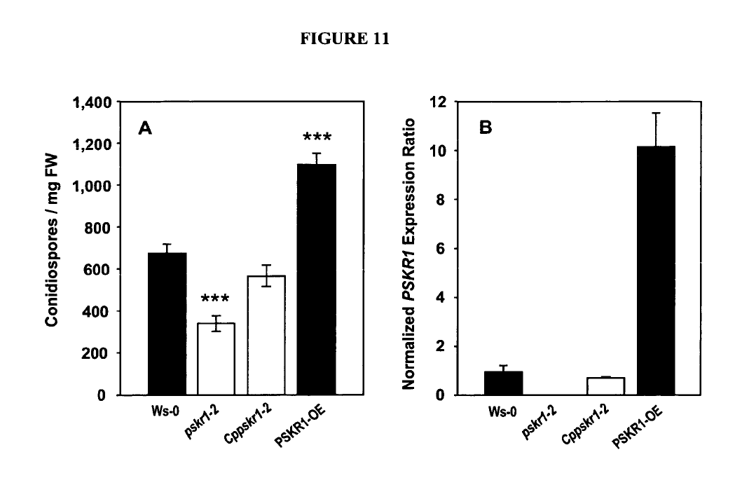

For overexpression of the gene, 3,060 by of the coding region including Start and Stop codons were amplified from genomic DNA, cloned into the Gateway destination vector pH2GW7 (Karimi et al., 2002), and mobilized into Arabidopsis by Agrobacterium-mediated transformation. The pathogen assays were performed as described before. All experiments were repeated 3 times and gave similar results (see FIG. 11A). Relative PSKR1 transcript accumulations in Arabidopsis seedlings (15 days after sowing) were determined by quantitative real time RT-PCR. Expression ratios were calculated using the 2^{-(ΔΔCT)} method with UBP22 (At5g10790) for normalization and wild-type PSKR1 expression as the reference. The bars (±SD) represent mean values of three technical replicates (see FIG. 11B).

### Results:

The PSKR expression was analyzed in mutant plants overexpressing PSKR. As shown in FIG. 11, the overexpression of PSKR1 (line PSKR1-OE) increases downy mildew susceptibility by almost 100%. Therefore, downy mildew susceptibility correlates with PSKR1 expression.

### Example 9

### The Increased Resistance Phenotype of pskr1 Mutants is not Due to Increased Defense Mechanisms

Marker genes for salicylic acid (SA)-, jasmonic acid (JA)-, and ethylene (JA/ethylene)-mediated signaling pathways were PR1a (At2g14610) PDF1.2 (At5g44420), and PR4 (At3g04720), respectively. Expression of these defense-related genes was analyzed by quantitative real-time RT-PCR in wild type (Ws), mutant (pskr1-2), and transgenic PSKR1 overexpressor (PSKR1-OE) plants upon spray treatment of cotyledons with water, or with conidiospore suspensions (40,000 spores/ml) of the H. arabidopsidis isolate Emwa1. Samples for RNA extraction and qRT-PCR were prepared at time point 0, and 24, 48, 72, and 120 hours after onset of treatment. Relative quantities of marker gene transcripts were normalized with AtOXA1 (At5g62050) and AtUBP22 (At5g10790) using the Q-Base software. Represented are means (±SD) from 3 technical replicates. Two independent experiments gave similar results.

### Results:

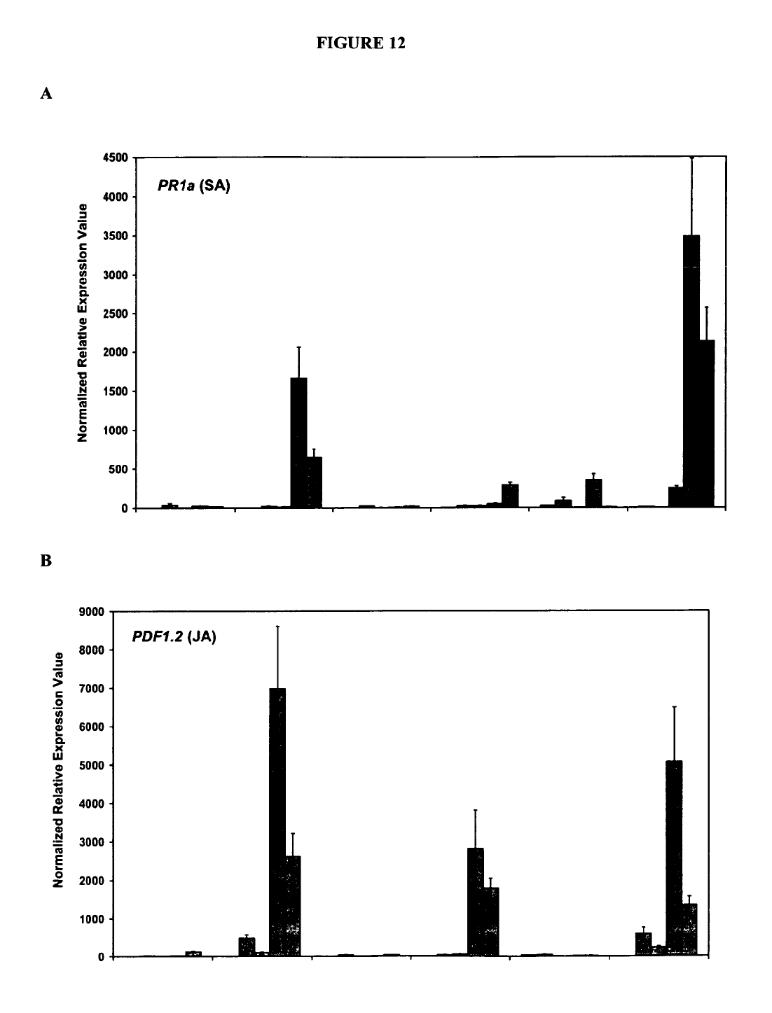

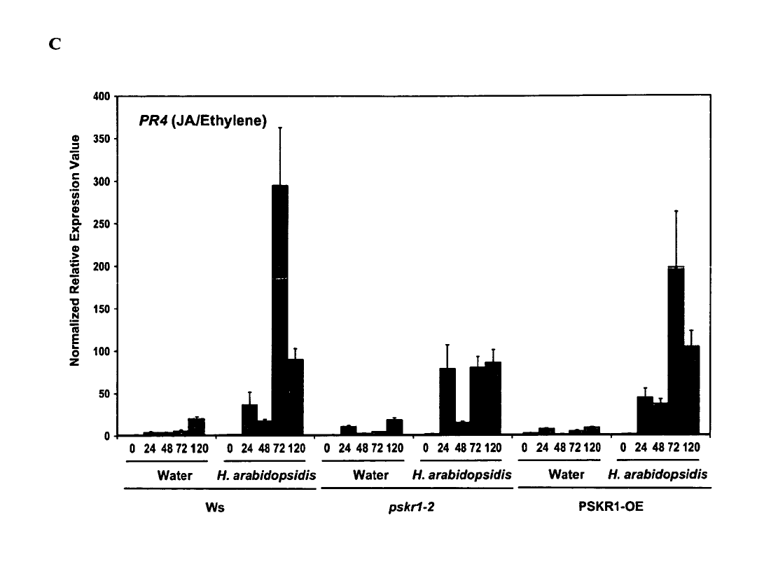

As shown in FIG. 12, the activation of SA-, JA-, and JA/ethylene-mediated defense signaling pathways in Arabidopsis is independent of PSKR1. The pskr1-2 mutant and PSKR overexpressing plants are not altered in these defense signaling pathways, i.e. increased resistance of the pskr1-2 mutant does not correlate with increased defense, and increased susceptibility of the overexpressing line is not correlated with decreased defense. A rather decreased defense activation in H. arabidopsidis-inoculated pskr1-2 mutant plants reflects most likely reduced downy mildew development.

### Example 10

### PSKR1 Suppression Causes Reduced Pathogen Proliferation

Pskr1 mutants were produced as disclosed in Example 4. These plants show delayed disease development in comparison to wild-type plants.

In a further set of experiments (see FIG. 13), the inventors have investigated whether such reduced susceptibility results from a reduced pathogen proliferation. To that purpose, wild-type plants, pskr1 mutants, the overexpressor line, and the complemented line were submitted to inoculations with three different pathogens: R. solanaearum (FIGS. 13A and 13B), H. arabidopsidis (FIG. 13C) and M. incognita (FIG. 13D).

Analysis of R. solanaearum proliferation (FIGS. 13A and 13B)

Four week-old plants were root-inoculated with a solution containing 10⁷ bacteria per ml of the virulent bacterial isolate RD15. For analyzing bacterial internal growth R. solanaearum, the procedure described above was applied (see the legend to FIG. 7C in connection with Example 7). R. solanacearum was re-extracted at different time points after inoculation to determine pathogen titers. For each time point, triplicate assays were performed for each A. thaliana line.

Analysis of H. arabidopsidis proliferation (FIG. 13C)

Plants were spray-inoculated with 40,000 spores/ml and cotyledons were collected 5 days post inoculation. Intercellular growth and branching of H. arabidopsidis was microscopically analyzed by trypan blue-staining. Infected seedlings were covered with trypan blue solution (0,01% w/v in 10% phenol, 10% lactic acid, 10% water, 20% glycerol, and 50% ethanol, v/v), boiled for 3 min, stored at room temperature overnight, and bleached with chloral hydrate at 2.5 g/ml, before being mounted in 50% glycerol onto microscope slides, and photographed.

Analysis of M. incognita proliferation (FIG. 13D)

For morphological analyses, nematode-infected roots of pskr1-2, PSKR1-OE and wild-type plants (ecotype Ws) were fixed in 2% glutaraldehyde in 50 mM Pipes buffer (pH 6.9) on 7, 14 and 21 days post inoculation and then dehydrated and embedded in Technovit 7100 (Heraeus Kulzer, Wehrheim, Germany) as described by the manufacturer. Embedded tissues were sectioned (3 μm) and stained in 0.05% toluidine blue, mounted in Depex (Sigma) and microscopy was performed using bright field optics. Images were collected with a digital camera (Axiocam; Zeiss). Tissue sections through galls on 7 days post inoculation from pskr1-2 and PSKR1-OE showed no difference in gall and giant cells formation in comparison with control. At later stages of gall development (14 and 21 days post inoculation) the giant cells from pskr1-2 mutant plants were significantly smaller. For giant cell surface measurements, serial sections stained with toluidine blue were examined using the AxioVision V 4.8.1.0 software. Finally, giant cell development upon M. incognita infection was quantified on numerized micrographs taken from thin-sectioned, toluidine blue-stained roots isolated from the different lines. The three biggest giant cells per gall from at least 50 galls per phenotype were chosen for measurements. Galls from pskr1-2 mutant plants contain significantly smaller giant cells in comparison to control plants at 14 days post inoculation.

### Results:

FIG. 13 clearly shows that PSKR1 suppression causes reduced proliferation of the following pathogens: R. solanacearum (bacterium), H. arabidopsidis (oomycete), and M. incognita (nematode).

In particular, FIGS. 13A and 13B show that multiplication of the bacterium R. solanacearum is strongly reduced in the absence of PSKR1 in the pskr1-2 mutant. Bacterial multiplication is restored to the wild-type level upon introduction of a functional PSKR1 gene into the pskr1-2 genetic background (complemented line Cppskr1-2), and increased in a line overexpressing PSKR1 under the control of the constitutive 35S promoter (overexpressing line PSKR1-OE). In conclusion, bacterial multiplication is drastically reduced (˜1,000-fold) in the pskr1-2 mutant, restored in the complemented line, and increased (˜2-fold) in the overexpressing line.

FIG. 13C shows that the network and hyphal branching of the oomycete H. arabidopsidis is strongly reduced in the absence of PSKR1 in the pskr1-2 mutant, but becomes aberrant upon overexpression of PSKR1 in the PSKR1-OE line.

FIG. 13D shows that the reduced egg mass production by the nematode M. incognita is a consequence of reduced giant cell sizes in the absence of PSKR1.

### Example 11

### Generation of Mutant Solanum lycopersicum Lines for the PSKR1 Gene By the TILLING Strategy

The tomato SlPSKR sequence (SEQ ID NO: 67) was used as the target for a TILLING strategy to obtain tomato lines with an inactive PSKR1 protein with reduced susceptibility to plant pathogens.

The TILLING method is known per se in the art, including the preparation of genomic DNA, the generation of DNA pools and superpools, the targeted identification of single nucleotide exchanges, and the deconvolution steps to obtain individuals (see e.g., Piron et al., 2010).

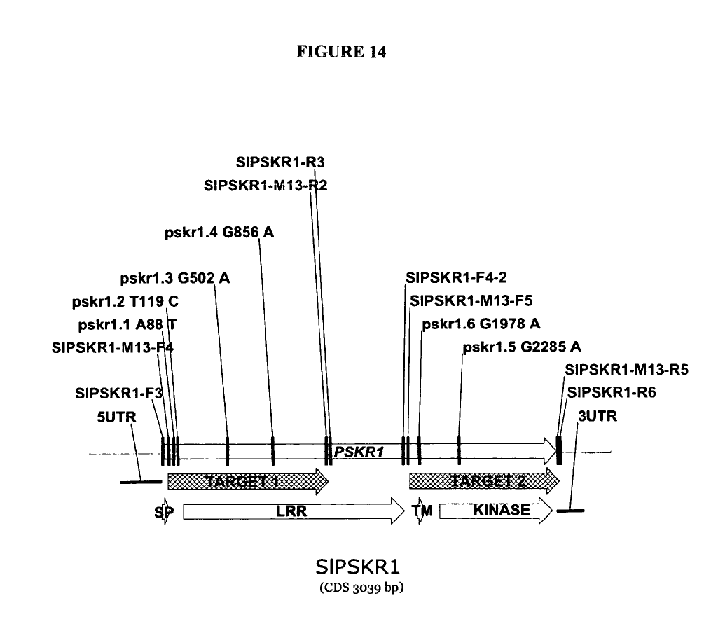

The inventors have tested plants from the parental M82 tomato line for interaction phenotypes with the oomycete, Phytophthora parasitica, and the root-knot nematode, Meloidogyne incognita. The parental line was fully susceptible to both pathogens. SlPSKR1 was selected as target gene for the TILLING approach, because it does not contain introns. The inventors have defined two genomic regions of SlPSKR1 to be targeted as shown in FIG. 14. The first target corresponds to the sequence coding for the extracellular LRR domain of the protein. The second target corresponds to the sequence coding for the C-terminal region of the protein, including membrane-spanning and kinase domains. Target 1 and 2 amplicons were generated with 2 sets of primers each, one set being specific for the target, and a second nested on the first and allowing to generate adaptors. Universal M13 primers that were labelled at the 5′end with the infra-red dyes IRD700 and IRD800 were used to generate the final amplicons that were analyzed for heteroduplexes after digestion by Endo1. Primers used for SlPSKR1 TILLING having sequences of SEQ ID NO: 76 to 85, are shown in the Table below:

| Target | Primer Name | Sequence 5Õ > 3Õ | Characteristics |
|---|---|---|---|
| 1. LRR domain | SIPSKR1-F3 | GGGTGTGTTGCAAGTTTGTGTGATC | Target-specific, PCR 1 |
| 1. LRR domain | SIPSKR1-R3 | CAAGTCTAACAGTTGCAGTTTTGAGC | Target-specific, PCR 1 |
| 1. LRR domain | SIPSKR1-M13-F4 | CACGACGTTGTAAAACGACTTACAAG | Generates adaptor, PCR 2 |
| | | CACAATCTC | |
| 1. LRR domain | SIPSKR1-M13-R2 | GGATAACAATTTCACACAGGCTGAGG | Generates adaptor, PCR 2 |
| | | AACAACTCC | |
| 2. TM-kinasedomain | SIPSKR1-F4-2 | GAGGGCAACCAAGGACTCTGCGGTG | Target-specific, PCR 1 |
| 2. TM-kinase domain | SIPSKR1-R6 | GCAGGACATCCGCTGGAAATATAAG | Target-specific, PCR 1 |
| 2. TM-kinase domain | SIPSKR1-M13-F5 | CACGACGTTGTAAAACGACCTGTCG | Generates adaptor, PCR 2 |
| | | AAATGCCAGC | |
| 2. TM-kinase domain | SIPSKR1-M13-R5 | GGATAACAATTTCACACAGGCTGAG | Generates adaptor, PCR 2 |
| | | GAACAACTCC | |
| Adaptor | M13F700 | CACGACGTTGTAAAACGAC | IRD700-labeled universal |
| Adaptor | M13R800 | GGATAACAATTTCACACAGG | IRD800-labeled universal |

The screen of 7×96-well titer plates containing genomic DNA from 8 individuals/well revealed 23 potential mutations in target 1 plus 14 potential mutations in target 2 (=37 potential mutants). The deconvolution procedure led to the identification of the first 6 individual lines, 4 harboring single nucleotide changes within target 1, and 2 with single nucleotide changes within target 2.

Seeds from the 6 individual lines were sown, to generate homozygous plants with reduced susceptibility to plant pathogens. Domains, targets, primer attachment sites, and the sites of the obtained 6 mutations within SlPSKR1 are indicated in FIG. 14.

### CONCLUSIONS

Altogether, the expression data and the phenotypical data indicate that PSK and PSKR genes are negative regulators of resistance to plant pathogens and that the enhanced resistance to pathogens and the reduced susceptibility to infection, which is observed in the PSK and PSKR knock-out mutants is due to a “loss of function” mutation in the PSK or PSKR gene.

Enhanced resistance of pskr1 mutants is not a consequence of constitutively activated, or pathogen-triggered defense responses and the mutants thus present a loss-of-susceptibility phenotype, rather than a gain of resistance. As shown in FIG. 12, the activation of salicylic acid (SA)-, jasmonic acid (JA)-, and ethylene (JA/ethylene)-mediated defense signaling pathways in Arabidopsis are independent of PSKR1. Marker genes for SA-, JA, and JA/ethylene-mediated signaling pathways were PR1a (At2g14610) PDF1.2 (At5g44420), and PR4 (At3g04720), respectively. Expression of these defense-related genes was analyzed by quantitative real-time RT-PCR in wild type (Ws), mutant (pskr1-2), and transgenic PSKR1 overexpressor (PSKR1-OE) plants upon spray treatment of cotyledons with water, or with conidiospore suspensions (40,000 spores/ml) of the H. arabidopsidis isolate Emwa1.

Moreover, data obtained with pskr1 mutants confirm a correlation between PSKR1 suppression and reduced pathogen proliferation.

Furthermore, data obtained with plants overexpressing PSK or PSKR confirm a correlation between expression of PSK and susceptibility to infection since overexpression of PSK or PSKR1 increases susceptibility to pathogen infection.

This is the first example ever found of a plant growth factor negatively regulating disease resistance.

### REFERENCES

Amano Y, Tsubouchi H, Shinohara H, Ogawa M, Matsubayashi Y. (2007). Tyrosine-sulfated glycopeptide involved in cellular proliferation and expansion in Arabidopsis. Proc Natl Acad Sci USA 104:18333-18338.
Bahyrycz A, Matsubayashi Y, Ogawa M, Sakagami Y, Konopinska D. (2005) Further analogues of plant peptide hormone phytosulfokine-alpha (PSK-alpha) and their biological evaluation. J Pept Sci 11:589-592.
Bahyrycz A, Matsubayashi Y, Ogawa M, Sakagami Y, Konopinska D. (2004). Plant peptide hormone phytosulfokine (PSK-alpha): synthesis of new analogues and their biological evaluation. J Pept Sci 10:462-469.
Bahyrycz A., Saniewska A., Konopinska D. Anti-pathogenic properties of plant peptide hormone phytosulfokines and its selected analogues. Pestycydy, 2008, (1-2), 109-117.
Chen Y F, Matsubayashi Y, Sakagami Y. (2000). Peptide growth factor phytosulfokine-alpha contributes to the pollen population effect. Planta 211:752-755.
Dangl J L, Holub E B, Debener T, Lehnackers H, Ritter C, Crute I R (1992). Genetic definition of loci involved in Arabidopsis-pathogen interactions. In Methods in Arabidopsis Research. Edited by Koncz C, Chua N-H, Schell J. Singapore: World Scientific Publishers:393-418
Elphinstone J G, Hennessy J, Wilson J K, Stead D E (1996). Sensitivity of different methods for the detection of Pseudomonas solanacearum in potato tuber extracts. EPPO/OEPP Bulletin 26:663-678.
Fields S, Song O (1989). A novel genetic system to detect protein-protein interactions. Nature 340: 245-246.
Fukuda H, Hirakawa Y, Sawa S. (2007). Peptide signaling in vascular development. Curr Opin Plant Biol 10:477-482.
Grefen C, Obrdlik P, Harter K. (2009). The determination of protein-protein interactions by the mating-based split-ubiquitin system (mbSUS). Methods Mol Biol 479:217-233.
Hanai H, Matsuno T, Yamamoto M, Matsubayashi Y, Kobayashi T, Kamada H, Sakagami Y. (2000). A secreted peptide growth factor, phytosulfokine, acting as a stimulatory factor of carrot somatic embryo formation. Plant Cell Physiol 41:27-32.
Hanai H, Nakayama D, Yang H, Matsubayashi Y, Hirota Y, Sakagami Y. (2000). Existence of a plant tyrosylprotein sulfotransferase: novel plant enzyme catalyzing tyrosine O-sulfation of preprophytosulfokine variants in vitro. FEBS Lett 470:97-101.
Hok S, Danchin EG, Allasia V, Panabières F, Attard A, Keller H. (2011). An Arabidopsis (malectin-like) leucine-rich repeat receptor-like kinase contributes to downy mildew disease. Plant Cell Environ, in press.
Igasaki T, Akashi N, Ujino-Ihara T, Matsubayashi Y, Sakagami Y, Shinohara K. (2003). Phytosulfokine stimulates somatic embryogenesis in Cryptomeria japonica. Plant Cell Physiol 44:1412-1416.
Jammes F, Lecomte P, de Almeida-Engler J, Bitton F, Martin-Magniette M L, Renou J P,

Abad P, Favery B. (2005). Genome-wide expression profiling of the host response to root-knot nematode infection in Arabidopsis. Plant J 44:447-458.
Kim B J, Gibson D M, Shuler M L. (2006). Effect of the plant peptide regulator, phytosulfokine-alpha, on the growth and Taxol production from Taxus sp. suspension cultures. Biotechnol Bioeng 95:8-14.
Komori R, Amano Y, Ogawa-Ohnishi M, Matsubayashi Y. (2009). Identification of tyrosylprotein sulfotransferase in Arabidopsis. Proc Natl Acad Sci U S A 106:15067-72.
Kutschmar A, Rzewuski G, Stührwohldt N, Beemster G T, Inz D, Sauter M. (2008). PSK-alpha promotes root growth in Arabidopsis. New Phytol 181:820-831.
Matsubayashi Y, Goto T, Sakagami Y. (2004). Chemical nursing: phytosulfokine improves genetic transformation efficiency by promoting the proliferation of surviving cells on selective media. Plant Cell Rep 23:155-158.
Matsubayashi Y, Hanai H, Hara O, Sakagami Y. (1996). Active fragments and analogs of the plant growth factor, phytosulfokine: structure-activity relationships. Biochem Biophys Res Commun 22:209-214.
Matsubayashi Y, Ogawa M, Kihara H, Niwa M, Sakagami Y. (2006). Disruption and overexpression of Arabidopsis phytosulfokine receptor gene affects cellular longevity and potential for growth. Plant Physiol 142:45-53.
Matsubayashi Y, Ogawa M, Morita A, Sakagami Y. (2002). An LRR receptor kinase involved in perception of a peptide plant hormone, PSK. Science 296:1470-72.
Matsubayashi Y, Sakagami Y. (1996). Phytosulfokine, sulfated peptides that induce the proliferation of single mesophyll cells of Asparagus officinalis L. Proc Natl Acad Sci USA 93:7623-7627.
Matsubayashi Y, Sakagami Y. (1999). Characterization of specific binding sites for a mitogenic sulfated peptide, phytosulfokine-alpha, in the plasma-membrane fraction derived from Oryza sativa L. Eur J Biochem.262:666-671.
Matsubayashi Y, Sakagami Y. (2000). 120- and 160-kDa receptors for endogenous mitogenic peptide, phytosulfokine-alpha, in rice plasma membranes. J Biol Chem 275:15520-15525.
Matsubayashi Y, Shinohara H, Ogawa M. (2006). Identification and functional characterization of phytosulfokine receptor using a ligand-based approach. Chem Rec 6:356-364.
Matsubayashi Y, Takagi L, Omura N, Morita A, Sakagami Y. (1999). The endogenous sulfated pentapeptide phytosulfokine-alpha stimulates tracheary element differentiation of isolated mesophyll cells of zinnia. Plant Physiol 120:1043-1048.
Matsubayashi Y, Takagi L, Sakagami Y. (1997). Phytosulfokine-alpha, a sulfated pentapeptide, stimulates the proliferation of rice cells by means of specific high- and low-affinity binding sites. Proc Natl Acad Sci USA 94:13357-13362.
Matsubayashi Y, Yang H, Sakagami Y. (2001). Peptide signals and their receptors in higher plants. Trends Plant Sci 6:573-577.
Matsubayashi Y. (2003). Ligand-receptor pairs in plant peptide signaling. J Cell Sci 116:3863-3870.
Motose H, Iwamoto K, Endo S, Demura T, Sakagami Y, Matsubayashi Y, Moore K L,
Fukuda H. (2009). Involvement of phytosulfokine in the attenuation of stress response during the transdifferentiation of zinnia mesophyll cells into tracheary elements. Plant Physiol 150:437-447.
Napier R. (2004). Plant hormone binding sites. Ann Bot 93:227-233.
Ohyama K, Ogawa M, Matsubayashi Y. (2008). Identification of a biologically active, small, secreted peptide in Arabidopsis by in silico gene screening, followed by LC-MS-based structure analysis. Plant J 55:152-160.
Ohyama K, Shinohara H, Ogawa-Ohnishi M, Matsubayashi Y. (2009). A glycopeptide regulating stem cell fate in Arabidopsis thaliana. Nat Chem Biol 5:578-580.
Piron F, Nicolaï M, Minoïa. S, Piednoir E, Moretti A, Salgues A, Zamir D, Caranta C,

Bendahmane A. (2010). An induced mutation in tomato eIF4E leads to immunity to two potyviruses. PLoS One 5:e11313.
Shinohara H, Matsubayashi Y. (2007). Functional immobilization of plant receptor-like kinase onto microbeads towards receptor array construction and receptor-based ligand fishing. Plant J 52:175-184.
Shinohara H, Ogawa M, Sakagami Y, Matsubayashi Y. (2007). Identification of ligand binding site of phytosulfokine receptor by on-column photoaffinity labeling. J Biol Chem 282:124-131.
Stagljar I, Korostensky C, Johnsson N, te Heesen S (1998) A genetic system based on split-ubiquitin for the analysis of interactions between membrane proteins in vivo. Proc Natl Acad Sci USA 95:5187-5192.
Toki S, Hara N, Ono K, Onodera H, Tagiri A, Oka S, Tanaka H. Early infection of scutellum tissue with Agrobacterium allows high-speed transformation of rice. Plant J. 2006 September;47(6):969-76. PubMed PMID: 16961734.
Yamakawa S, Matsubayashi Y, Sakagami Y, Kamada H, Satoh S. (1999). Promotive effects of the peptidyl plant growth factor, phytosulfokine-alpha, on the growth and chlorophyll content of Arabidopsis seedlings under high night-time temperature conditions. Biosci Biotechnol Biochem 63:2240-2243.
Yang H, Matsubayashi Y, Hanai H, Nakamura K, Sakagami Y. (2000). Molecular cloning and characterization of OsPSK, a gene encoding a precursor for phytosulfokine-alpha, required for rice cell proliferation. Plant Mol Biol 44:635-647.
Yang H, Matsubayashi Y, Hanai H, Sakagami Y. (2000). Phytosulfokine-alpha, a peptide growth factor found in higher plants: its structure, functions, precursor and receptors. Plant Cell Physiol 41:825-830.
Yang H, Matsubayashi Y, Nakamura K, Sakagami Y. (1999). Oryza sativa PSK gene encodes a precursor of phytosulfokine-alpha, a sulfated peptide growth factor found in plants. Proc Natl Acad Sci USA 96:13560-13565.
Yang H, Matsubayashi Y, Nakamura K, Sakagami Y. (2001). Diversity of Arabidopsis genes encoding precursors for phytosulfokine. Plant Physiol 127:842-851.
Yang H, Morita A, Matsubayashi Y, Nakamura K, Sakagami Y. (2000). A rapid and efficient system of Agrobacterium infection-mediated transient gene expression in rice Oc cells and its application for analysis of the expression and antisense suppression of preprophytosulfokine, a precursor of phytosulfokine-a, encoded by OsPSK gene. Plant Cell Physiol 41:811-816.

1-20. (canceled)

21. A method for protecting a plant against pathogens, comprising a step of inhibiting permanently or transiently phytosulfokine (PSK) function in said plant or an ancestor thereof.

22. The method of claim 21, wherein said method increases pathogen resistance or decreases pathogen proliferation in said plant or an ancestor thereof.

23. The method of claim 21, wherein said plant has a defective PSK gene, a defective PSK peptide, a defective PSK receptor (PSKR) gene and/or a defective PSKR receptor.

24. The method of claim 23, wherein said PSK or PSKR gene is defective as a result of a deletion, insertion and/or substitution of one or more nucleotides, site-specific mutagenesis, ethyl methanesulfonate (EMS) mutagenesis, targeting induced local lesions in genomes (TILLING), EcoTILLING, knock-out techniques, inactivation with a ribozyme or antisense nucleic acid, or by gene silencing induced by RNA interference.

25. The method of claim 24, wherein the PSK and/or PSKR gene(s) is/are fully or partially deleted or are silenced with RNAi.

26. The method of claim 23, wherein each copy of the PSK gene, when present in several copies in said plant cells, is rendered defective.

27. The method of claim 23, wherein the genes PSKR1 and PSKR2 are rendered defective in said plant cells.

28. The method of claim 21, wherein said plant pathogens are selected from fungi, oomycetes, nematodes or bacteria.

29. A method for producing a plant having increased resistance to plant pathogens, wherein the method comprises the following steps:
(a) inactivation of PSK and/or PSKR gene(s) in plant cells;
(b) optionally, selection of plant cells of step (a) with defective PSK and/or PSKR gene(s);
(c) regeneration of plants from cells of step (a) or (b); and
(d) optionally, selection of a plant of (c) with increased resistance to pathogens, said plant having defective PSK or PSKR gene(s).

30. The method of claim 29, wherein, in step (a), said PSK or PSKR gene is inactivated by deletion, insertion and/or substitution of one or more nucleotides, site-specific mutagenesis, ethyl methanesulfonate (EMS) mutagenesis, targeting induced local lesions in genomes (TILLING), EcoTILLING, knock-out techniques, or by gene silencing induced by RNA interference.

31. The method of claim 29, wherein the plant is a dicot or a monocot.

32. The method of claim 29, wherein said monocots and dicots are selected from the families Solanaceae, Liliaceae, Apiaceae, Chenopodiaceae, Vitaceae, Fabaceae, Cucurbitaceae, Brassicacea or Poaceae.

33. A method of identifying a molecule that modulates the PSKR gene expression, the method comprising:
(a) providing a cell comprising a nucleic acid construct that comprises a PSKR gene promoter sequence operably linked to a reporter gene;
(b) contacting the cell with a candidate molecule;
(c) measuring the activity of PSKR promoter by monitoring of the expression of a marker protein encoded by the reporter gene in the cell;
(d) selecting a molecule that modulates the expression of the marker protein.

34. The method of claim 33, wherein the molecule inhibits the expression of PSKR.

35. A modified plant having increased pathogen resistance, wherein said plant is from the families Solanaceae, Liliaceae, Apiaceae, Chenopodiaceae, Vitaceae, Fabaceae, Cucurbitaceae, Brassicacea or Poaceae, and wherein said plant has a defective PSKR1 gene.

36. The plant of claim 35, wherein said plant is a tomato plant having a defective PSKR1 gene.
